(19) United States
(12) Patent Application Publication
(10) Pub. No.: US 2021/0401867 A1
(43) Pub. Date: Dec. 30, 2021

(54) PHOTOSENSITIZERS AND METHOD OF TREATING CANCER USING THE SAME

(71) Applicants: Cheng-Wen Wu, Taipei (TW); NanoRay Biotech Co., Ltd., Taipei (TW)

(72) Inventors: CHENG-WEN WU, Taipei (TW); ERH-HSUAN LIN, Taipei (TW); WAN-TING TSENG, Taipei (TW)

(21) Appl. No.: 16/612,414

(22) PCT Filed: Apr. 1, 2019
(86) PCT No.: PCT/US2019/025103

§ 371 Date: Nov. 11, 2019

Related U.S. Application Data
(60) Provisional application No. 62/683,009, filed on Jun. 10, 2018.

(51) Int. Cl. A61K 31/7072; A61N 5/10; A61K 41/00

(52) U.S. Cl. CPC A61K 31/7072; A61N 2005/1098; A61K 41/0038; A61N 5/10

(57) ABSTRACT

The present disclosure provides a heavy atom carrier and a method of treating cancer using the heavy atom carriers in conjunction with monochromatic X-ray. The heavy atom carriers are halogen-containing heavy atom carriers.

### CROSS-REFERENCE TO RELATED APPLICATION

The present application claims priority to U.S. Provisional Application No. 62/683,009, filed on Jun. 10, 2018, the entirety of which is incorporated herein by reference.

### FIELD

The present disclosure generally relates to a method of treating cancer using a photosensitizer and a monochromatic X-ray.

### BACKGROUND OF THE DISCLOSURE

Conventional cancer therapies are mostly non-specific treatments including surgical resection, chemotherapy, and radiation therapy. Although some non-specific treatments may prolong the overall length of survival of the treated patients, adverse effects associated with those treatments may cause significant deterioration of life quality of the patients. Physicians have attempted to optimize the therapeutic regimens of non-specific treatments, aiming to improve the clinical outcome and reduce adverse effects. However, conventional surgical resection, chemotherapy, and radiation therapy are confronted with many obstacles. Success rate of tumor resection has been disease stage and surgeon dependent; it has been difficult to detect possible local invasions by referencing to limited number of pathological samples. Systemic chemotherapy has shown limited improvements in overall length of survival, and usually led to severe adverse effects that reduce life quality of the patients. Radiation therapy has been used to treat localized targets, but is only curative for a limited number of cancer types.

In contrast to non-specific treatments, cancer-specific treatments selectively recognize and kill cancer cells. For example, “targeted chemotherapy” combines cancer-specific antibodies with known chemotherapeutic agents to identify over-expressed biomarkers on cancer cells and thus selectively kill the identified cancer cells. However, existing targeted chemotherapies have exhibited limited effectiveness in improving overall length of survival. In addition, adverse effects of existing targeted chemotherapies include photosensitivity, rash, itching, or change of hair color and skin color.

Another example of cancer-specific treatment is an Auger molecular therapy. Conventional Auger molecular therapies utilize photosensitizers that contain specific element(s) that can be photosensitized by X-ray irradiation. However, some photosensitizers cannot effectively enter cancer cells, and may induce non-selective cytotoxicity, which causes DNA damages in normal cells.

Therefore, there is a need for an improved cancer-specific treatment. There is also a need for a novel agent for use in an Auger molecular therapy that are cancer specific but are cytotoxicity-free to normal cells.

### SUMMARY OF THE DISCLOSURE

In view of the shortcomings in the art, it is an objective of the present disclosure to provide one or more novel agents for use in the Auger molecular therapy that are cancer specific but are cytotoxicity-free to normal cells.

It is another objective of the present disclosure to provide a panel of photosensitizers and methods of treating cancer using the photosensitizers in conjunction with monochromatic X-ray.

Preferably, the photosensitizers are heavy atom carriers. Specifically, the heavy atom carriers can be halogen-containing heavy atom carriers.

An embodiment of the present disclosure provides an Auger molecular therapy, comprising steps of: a) administering a pharmaceutical composition comprising a therapeutically effective dosage of the heavy atom carrier to a subject; and b) irradiating a monochromatic X-ray to the subject.

An embodiment of the present disclosure provides a method for treating cancer in the subject, comprising steps of: a) administering a pharmaceutical composition comprising a therapeutically effective dosage of iododeoxyuridine (IdU) to the subject; and b) irradiating the monochromatic X-ray to the subject.

An embodiment of the present disclosure provides a combination of the pharmaceutical composition comprising a therapeutically effective dosage of IdU and the radiation dose of the monochromatic X-ray for use in the treatment of cancer.

An embodiment of the present disclosure provides a kit. The kit comprises a) the pharmaceutical composition comprising the therapeutically effective dosage of IdU; and b) the monochromatic X-ray device.

An embodiment of the present disclosure provides a use of a combination of the pharmaceutical composition comprising the therapeutically effective dosage of IdU and the radiation dose of the monochromatic X-ray for the manufacture of a medicament for the treatment of cancer.

In a preferred embodiment, the pharmaceutical composition further comprises a pharmaceutical carrier for facilitating absorption and distribution in the subject.

In a preferred embodiment, IdU is administered to the subject through a local route or a systemic route.

In a preferred embodiment, the local route is a topical route, an intratumoral injection, or an intravascular injection.

In a preferred embodiment, the systemic route is a gastrointestinal route, an intravenous injection, an intravenous drip infusion, an intramuscular injection, or a subcutaneous injection.

In a preferred embodiment, the radiation dose of the monochromatic X-ray irradiated to the subject is 1 Gy-9 Gy.

In a preferred embodiment, the radiation dose of the monochromatic X-ray irradiated to the subject is 4.5 Gy or 9 Gy.

In a preferred embodiment, the therapeutically effective dosage of IdU is an injection amount of 3.75 mg-62.5 mg.

In a preferred embodiment, the therapeutically effective dosage of IdU is an injection volume of 7.5 ml-125 ml.

In a preferred embodiment, the monochromatic X-ray has an energy of 33 keV.

### BRIEF DESCRIPTION OF THE DRAWINGS

Implementations of the present technology will now be described, by way of example only, with reference to the attached figures.

FIG. 1 is a spectrum of NanoRay radiation (NR) when the photon energy is 14 keV in accordance with an exemplary embodiment of the present disclosure.

FIG. 2 is a spectrum of NR when the photon energy is 33.4 keV in accordance with an exemplary embodiment of the present disclosure.

FIG. 3 is a spectrum of RS2000 X-ray in accordance with an exemplary embodiment of the present disclosure.

FIG. 4 is an in vitro cell viability chart of FaDu cells when treated with 0 μM, 1 μM, 5 μM, 10 μM, 20 μM, and 40 μM BrdU, in accordance with an exemplary embodiment of the present disclosure.

FIG. 5 is an in vitro cell viability chart of FaDu cells when treated with 0 μM, 2.84 μM, 5.65 μM, 14.12 μM, and 28.24 μM IdU, in accordance with an exemplary embodiment of the present disclosure.

FIG. 6 is an in vitro cell viability chart of FaDu cells when irradiated by 1 Gy, 2 Gy, and 4 Gy of 33 keV NR, in accordance with an exemplary embodiment of the present disclosure.

FIG. 7A is a flow cytometry analysis of FaDu cells without BrdU treatment; FIG. 7B shows a flow cytometry analysis of FaDu cells after 24 hours of incubation with 0.5 μM BrdU; FIG. 7C is a flow cytometry analysis of FaDu cells after 24 hours of incubation with 1 μM BrdU; FIG. 7D is a flow cytometry analysis of FaDu cells after 24 hours of incubation with 10 μM BrdU, in accordance with exemplary embodiments of the present disclosure.

FIG. 8A is a flow cytometry analysis of FaDu cells without IdU treatment; FIG. 8B is a flow cytometry analysis of FaDu cells after 24 hours of incubation with 1 μM IdU; FIG. 8C is a flow cytometry analysis of FaDu cells after 24 hours of incubation with 5 μM IdU; FIG. 8D is a flow cytometry analysis of FaDu cells after 24 hours of incubation with 10 μM IdU, in accordance with exemplary embodiments of the present disclosure.

FIG. 9A is an in vitro cell viability chart of FaDu cells after treated with 1 μM BrdU, 1 Gy of 14 keV NR, and 1 μM BrdU with 1 Gy of 14 keV NR; FIG. 9B are colony forming assay results of FaDu cells after the same treatment scheme as in FIG. 9A; FIG. 9C is a bar chart representing the colony forming assay results in FIG. 9B, in accordance with exemplary embodiments of the present disclosure.

FIG. 10A is an in vitro cell viability chart of FaDu cells after treated with 5 μM IdU, 10 μM IdU, 1 Gy of 33 keV NR, 5 μM IdU with 1 Gy of 33 keV NR, and 10 μM IdU with 1 Gy of 33 keV NR; FIG. 10B are colony-forming assay results of FaDu cells after the same treatment scheme as in FIG. 10A; FIG. 10C is a bar chart representing the colony forming assay results in FIG. 10B, in accordance with exemplary embodiments of the present disclosure.

FIG. 11A is an in vitro cell viability chart of FaDu cells after treated with 5 μM IdU, 10 μM IdU, 2 Gy of 33 keV NR, 5 μM IdU with 2 Gy of 33 keV NR, and 10 μM IdU with 2 Gy of 33 keV NR; FIG. 11B are colony forming assay results of FaDu cells after the same treatment scheme as in FIG. 11A; FIG. 11C is a bar chart representing the colony forming assay results in FIG. 11B, in accordance with exemplary embodiments of the present disclosure.

FIG. 12 is a schematic diagram of an in vivo animal experiment with IdU and NR, in accordance with exemplary embodiments of the present disclosure.

FIG. 13A is a schematic diagram of an in vivo animal experiment for determining an injection concentration of IdU; FIG. 13B are flow cytometry results to determine the injection concentration of IdU; FIG. 13C shows IdU incorporation rates, in accordance with exemplary embodiments of the present disclosure.

FIG. 14 is a tumor growth chart in an in vivo animal experiment after treated with IdU and NR, in accordance with exemplary embodiments of the present disclosure.

FIG. 15A is a tumor growth index chart of the FaDu cells after treated with 2.25 Gy of NR, 50 μg of IdU, or 2.25 Gy of NR and 50 μg of IdU; FIG. 15B is the tumor growth index chart of the FaDu cells after treated with 2.25 Gy of RS2000, 50 μg of IdU, or 2.25 Gy of RS2000 and 50 μg of IdU; FIG. 15C is the tumor growth index chart of the FaDu cells after treated with 4.5 Gy of NR, 50 μg of IdU, or 4.5 Gy of NR and 50 μg of IdU; FIG. 15D is the tumor growth index chart of the FaDu cells after treated with 4.5 Gy of RS2000, 50 μg of IdU, or 4.5 Gy of RS2000 and 50 μg of IdU; FIG. 15E is the tumor growth index chart of FaDu cells after treated with 9 Gy of NR, 50 μg of IdU, or 9 Gy of NR and 50 μg of IdU; FIG. 15F is the tumor growth index char of FaDu cells treated with 9 Gy of RS2000, 50 μg of IdU, or 9 Gy of RS2000 and 50 μg of IdU, in accordance with exemplary embodiments of the present disclosure.

FIG. 16A is a qualitative result of an immunohistochemical (IHC) examination 1 day after the FaDu cells treated with 50 μg of IdU, 4.5 Gy of NR, 4.5 Gy of RS2000, 50 μg of IdU and 4.5 Gy of RS2000, and 50 μg of IdU and 4.5 Gy of IdU; FIG. 16B is a quantitative result of an IHC examination 1 day after the FaDu cells treated with 50 μg of IdU, 4.5 Gy of NR, 4.5 Gy of RS2000, 50 μg of IdU and 4.5 Gy of RS2000, and 50 μg of IdU and 4.5 Gy of IdU, in accordance with exemplary embodiments of the present disclosure.

FIG. 17A is a Western blotting result for protein expression level of phosphor-ATM (pATM), γH2AX, and c-CASP3 in FaDu cells 30 minutes after being treated with 10 IdU, 2 Gy of RS2000, 2 Gy of RS2000 and 10 μM IdU, 2 Gy of NR, and 2 Gy of NR and 10 μM IdU; FIG. 17B is a Western blotting result for protein expression level of phosphor-ATM (pATM), γH2AX, and c-CASP3 in FaDu cells 3 days after being treated with 10 μM IdU, 2 Gy of RS2000, 2 Gy of RS2000 and 10 μM IdU, 2 Gy of NR, and 2 Gy of NR and 10 μM IdU, in accordance with exemplary embodiments of the present disclosure.

FIG. 18 is a chemical structure of dibromocurcumin (DBC) in accordance with an exemplary embodiment of the present disclosure.

FIG. 19A is an in vitro cell viability chart of FaDu cells after treated with 10 dibromocurcumin (DBC), 20 μM DBC, 40 μM DBC, 80 μM DBC, 120 μM DBC, and 160 μM DBC; FIG. 19B is a bar chart for colony forming assay of FaDu cells after the same treatment scheme as in FIG. 19A, in accordance with exemplary embodiments of the present disclosure.

FIG. 20A is an in vitro cell viability chart of FaDu cells after treated with 1 Gy of 14 keV NR, 10 μM DBC, 20 μM DBC, 10 μM DBC with 1 Gy of 14 keV NR, and 20 μM DBC of 14 keV NR; FIG. 20B is a bar chart for colony forming assay of FaDu cells after the same treatment scheme as in FIG. 20A, in accordance with exemplary embodiments of the present disclosure.

FIG. 21A is an in vitro cell viability chart of FaDu cells after treated with 1 μM BrdU, 10 μM IdU, 1 Gy conventional X-ray, 1 μM BrdU with 1 Gy conventional X-ray, 2 Gy conventional X-ray, and 10 μM IdU with conventional X-ray; FIG. 21B are colony forming assay results of FaDu cells after the same treatment scheme as in FIG. 21A; FIG. 21C is a bar chart representing the colony forming assay results in FIG. 21B, in accordance with exemplary embodiments of the present disclosure.

FIG. 22A are comet assay results of FaDu cells after treated with 1 μM BrdU, 1 Gy of conventional X-ray, 1 μM BrdU with conventional X-ray, 1 Gy of 14 keV NR, and 1 μM BrdU with 1 Gy of 14 keV NR; FIG. 22B is a bar chart representing the comet assay results of FIG. 22A, in accordance with exemplary embodiments of the present disclosure.

FIG. 23A is a plurality of p-γH2AX immunofluorescent staining results for FadU cells after treated with 1 μM BrdU, 1 Gy of conventional X-ray, 1 μM BrdU with conventional X-ray, 1 Gy of 14 keV NR, and 1 μM BrdU with 1 Gy of 14 keV NR; FIG. 23B shows a bar chart representing the immunofluorescent staining of FIG. 23A, in accordance with exemplary embodiments of the present disclosure.

### DETAILED DESCRIPTION OF THE PREFERRED EMBODIMENTS

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

According to an exemplary embodiment of the present disclosure, a method of treating cancer includes the steps of: (S1) administrating a pharmaceutical composition comprising a therapeutically effective dosage of a heavy atom carrier to a subject; and (S2) irradiating an electromagnetic radiation to the subject.

According to an exemplary embodiment of the present disclosure, a combination of the pharmaceutical composition comprising the therapeutically effective dosage of the heavy atom carrier and a radiation dose of the electromagnetic radiation for use in the treatment of cancer.

According to an exemplary embodiment of the present disclosure, a kit comprising the pharmaceutical composition and the electromagnetic radiation device is provided. The pharmaceutical composition comprises the therapeutically effective dosage of the heavy atom carrier.

According to an exemplary embodiment of the present disclosure, a use of a combination of the pharmaceutical composition and the radiation dose of the electromagnetic radiation for the manufacture for the treatment of cancer is provided. The pharmaceutical composition comprises the therapeutically effective dosage of the heavy atom carrier.

The therapeutically effective dosage refers to an amount effective, for periods of time necessary, to achieve the desired therapeutic result, such as suppression or inhibition of tumor growth. The therapeutically effective dosage of the heavy atom carrier may vary according to factors such as the tumor size, age, gender, and weight of the subject, and the ability of the combination of the heavy atom carrier and the electromagnetic radiation to elicit a desired response in the subject. Dosage regimens may be adjusted to provide an optimum therapeutic response. The therapeutically effective dosage is also one in which any toxic or detrimental effects of the heavy atom carrier and/or the electromagnetic radiation are outweighed by therapeutically beneficial effects.

In at least one exemplary embodiment, the method is an Auger molecular therapy (AMT), which is a consequence of Auger effects on the cancer cells. The Auger effect is a process generated from a heavy element atom when being irradiated by monochromatic X-rays. If an electron in the K-shell of heavy element atom is removed, another electron in the outer shell of heavy element atom will move downward to fill the vacancy, and the downward movement of outer-shell electrons may generate an energy. The energy is then transferred to the outer shell and causes electrons to be ejected from the outer shell of the heavy element atom. The ejected electrons are Auger electrons. The AMT utilizes the Auger electrons to damage intracellular components in the cancer cells. The heavy atom carrier is one of the photosensitizers, and comprises one or more heavy element atoms. The heavy atom carrier in the AMT generates the Auger electrons through NR irradiation. The AMT exhibits significant cancer treatment outcome and minimizes damage to normal tissue. The method may be used to treat malignant solid tumors, such as head and neck cancer, breast cancer, lung cancer, colon cancer, esophagus cancer, hepatoma, melanoma, prostate cancer, or treat squamous cell carcinoma (SCC) or adenocarcinoma (AD) from various part of the body. The method may also be used to treat leukemia.

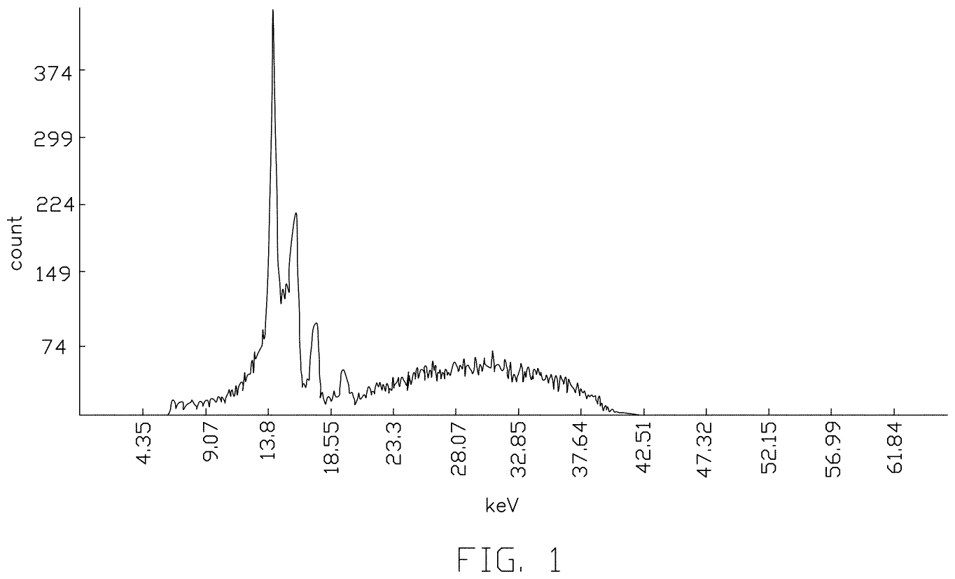

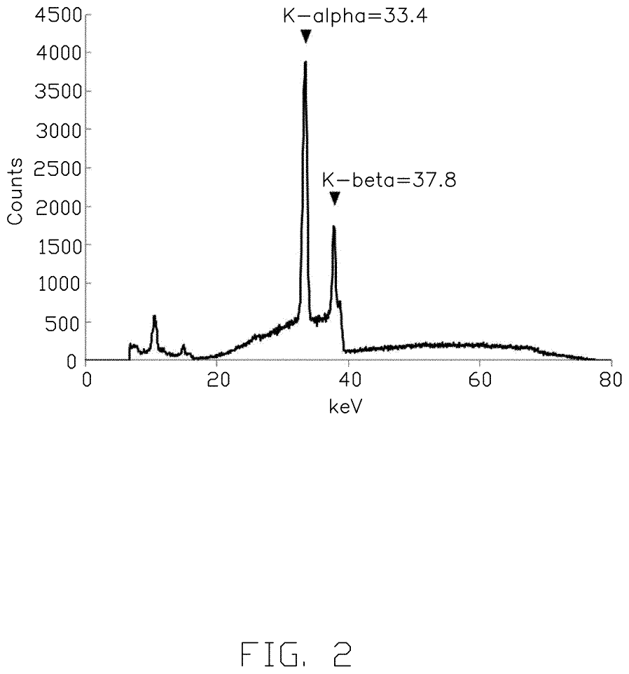

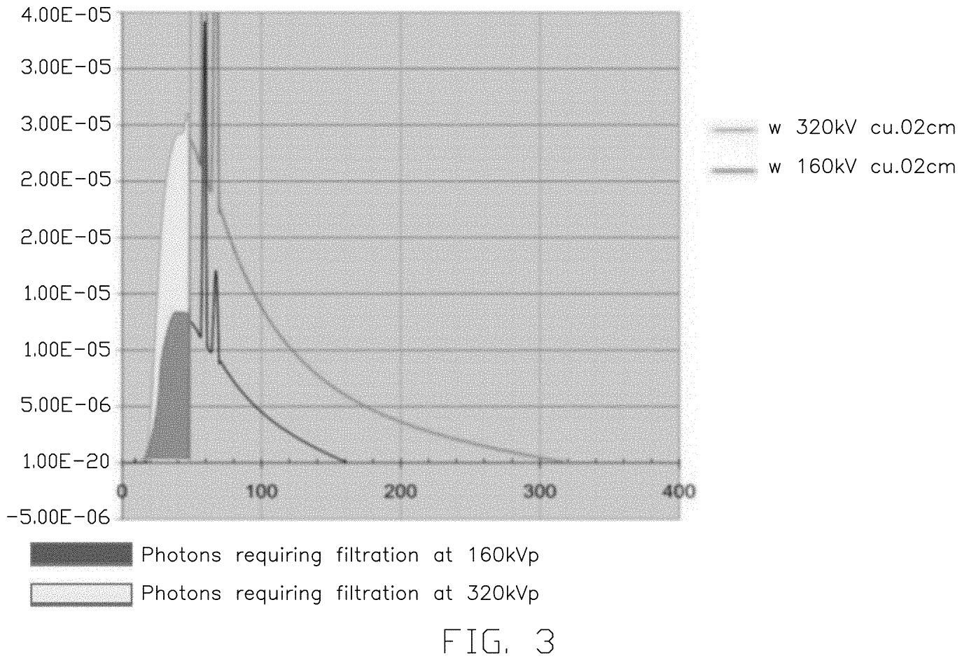

The electromagnetic radiation administered to the subject may be a monochromatic X-ray. In an exemplary embodiment, the monochromatic X-ray may be NanoRay radiation (NR), which has a photon energy similar to X-ray but a full width at half maximum (FWHM) narrower than X-ray. Referring to FIG. 1-3, spectrums of NR and RS2000 are provided in accordance with exemplary embodiments of the present disclosure. FIG. 1 and FIG. 2 show the spectrums of NR have peaks on 14 keV and 33 keV. To compare spectrums of the monochromatic X-ray and a conventional X-ray, FIG. 3 shows the spectrum of a conventional X-ray source RS2000. RS2000 is an ionizing irradiator producing Bremsstrahlung radiation for conventional radiation therapy. RS2000 also has a spectrum that covers 14 keV and 33 keV, but the photon energy on 14 keV and 33 keV in RS2000 is lower than NR. FIG. 1-3 demonstrates that NR has a more concentrated photon energy than conventional X-ray. Therefore, NR can excite photosensitizers more efficiently with lower radiation doses.

In the exemplary embodiment, the heavy atom carrier generates the Auger electrons upon irradiation by the electromagnetic radiation. The heavy atom carrier contains halogen, such as bromide (Br) or iodide (I), and/or heavy elements, such as Pt, Ca, Ti, Gd, Y, Ru Co, Se, Kr, Sr, Mo, Rh, Pd, Ag, Cd, Sn, Xe, Ba, Ta, W, Re, Os, Ir, Au, Hg, Tl, Pb, Th, U, lanthanides, or other heavy elements that promote generation of the Auger electrons in AMTS. Specifically, the halogen-containing heavy atom carriers may be iododeoxyuridine (IdU), bromodeoxyuridine (BrdU), dibromocurcumin (DBC) or 4,5,6,7-Tetrachloro-3′,6′-dihydroxy-2′,4′,5′,7′-tetraiodo-3H-spiro [ isobenzofuran-1,9′-xanthen]-3-one (rose Bengal; RB). The heavy atom carriers of various exemplary embodiments of the present disclosure exhibit pharmacokinetic characteristics suitable for clinical applications, suggesting that the heavy atom carriers can effectively enter and distribute among tumor cells. Furthermore, metabolites of the heavy atom carriers exhibit low toxicity and preferred excretion or elimination rate.

The pharmaceutical composition administered to the subject may further comprise a pharmaceutical carrier that facilitates absorption and distribution of the heavy atom carriers in the subject. In some exemplary embodiments, the pharmaceutical carriers may be liposomes, nanofibers, protein conjugates, dendrimers, or microspheres (e.g., a biodegradable polymer poly (lactic-co-glycolic) acid microsphere). A therapeutically effective dosage of the pharmaceutical composition administered to the subject is sufficient to induce statistically significant cytotoxicity to cancer cells, when comparing to subjects receiving only conventional X-ray or NRs in the following examples.

The administration route for the pharmaceutical composition may be a local route or a systemic route. The local route comprises a topical route, an intratumoral injection, or an intravascular injection. The systemic route comprises a gastrointestinal route, an intravenous injection, an intravenous drip infusion, an intramuscular injection, or a subcutaneous injection. The administration route may vary according to location of the cancer cells, properties of the pharmaceutical composition, or compliance of the subject to the method.

The dose of the electromagnetic radiation may vary according to weight of the subject, ability of the heavy atom carrier in generating the Auger electrons, or type of the electromagnetic radiation being used. The photon energy of the electromagnetic radiation may also vary according to the heavy atom carrier being administered, the weight of the subject, or the mass or location of the cancer cells. The dose of the electromagnetic radiation administered to the cancer cells of the subject is sufficient to induce generation of the Auger electrons by the heavy atom carrier.

Examples of using halogen-containing or heavy element-containing heavy atom carrier in treating cancer in accordance with the exemplary embodiments of the present disclosure are described as follows. Specifically, Examples 1 is administering BrdU and IdU in conjunction with the monochromatic X-ray; Example 2 is administering DBC in conjunction with the monochromatic X-ray; Example 3 is administering BrdU or IdU with conventional X-ray; and Example 4 compares the therapeutic effect between monochromatic X-ray and conventional X-ray.

1. Combining BrdU or IdU with Monochromatic X-Ray in Auger Molecular Therapy

Both of bromodeoxyuridine (BrdU) and iododeoxyuridine (IdU) comprise uridine, which would incorporate in DNAs of the cancer cells to cause DNA double strand breaks (DNA DSB). The DNA DSB is a major cause of cell death among cancer cells. The Auger electrons generated from the heavy atom carriers when irradiated by NanaoRay (NR) have an average linear energy transfer (LET) of around 100 keV/μm, and the average separation between ionizing events coincides with the diameter of DNA double helix (2 nm). Therefore, NR may mediate DNA DSB when the cancer cells are incorporated with BrdU or IdU. The combination of NR and BrdU or IdU is a promising cancer therapy.

1.1 Combining BrdU or IdU with Monochromatic X-Ray In Vitro

In the following examples, FaDu cells are used in cell viability tests and colony forming assays. FaDu cells (ATCC HTB-43) is a human head-and-neck cell line with squamous phenotype. FaDu cells used in the following examples are cultured in DMEM medium supplemented with 10% fetal bovine serum (FBS) and 1% penicillin/streptomycin, in a humidified incubator maintained at 37° C. and 5% CO₂.

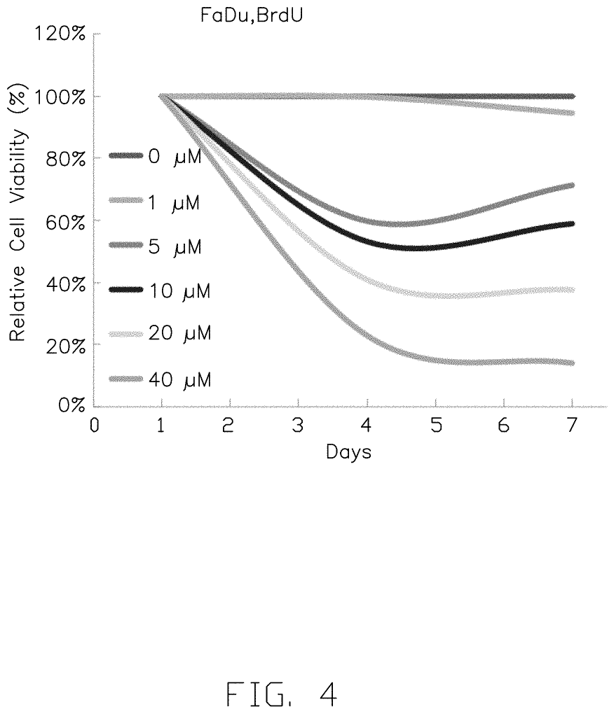

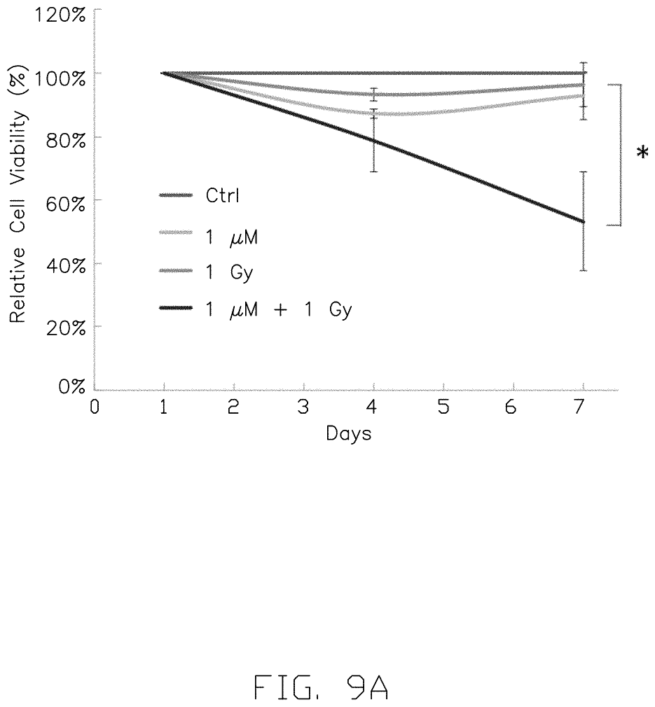

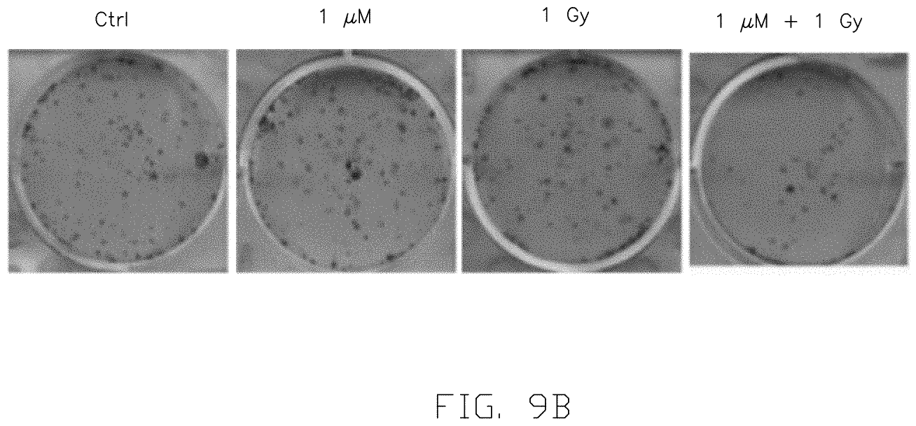

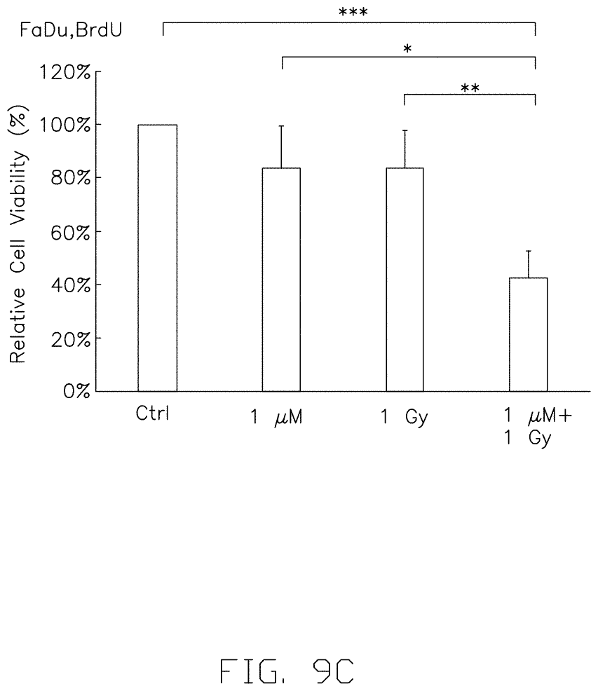

In FIGS. 4-8D, different dosages of BrdU and IdU, and different radiation doses of NR are used to determine one or more effective dosages of BrdU, IdU, and NR when used alone. In FIGS. 9A-11C, different dosage combinations of BrdU and NR, and IdU and NR are used to evaluate the combined therapeutic effects of monochromatic X-ray. The control groups (Ctrl) in FIGS. 4-8D are cells without BrdU, IdU, and NR treatments.

Referring to FIG. 4, an in vitro cell viability chart of FaDu cells when treated with BrdU is provided in accordance with an exemplary embodiment of the present disclosure. FaDu cells are treated with different dosages of BrdU for 24 hours and then FaDu cells are analyzed daily for cell viability. FaDu cells are derived from pharynx squamous cell carcinoma of human, and are resistant to radiation. 3×10⁵ FaDu cells are seeded in 6-cm-plates. After 24 hours, different dosages of BrdU are added into the culture medium and incubated with the FaDu cells for another 24 hours. FIG. 4 shows that the cell viability is less than 50% when treated with less than 10 μM BrdU on Day 7. When FaDu cells are treated with 1 μM, BrdU demonstrates almost no toxicity.

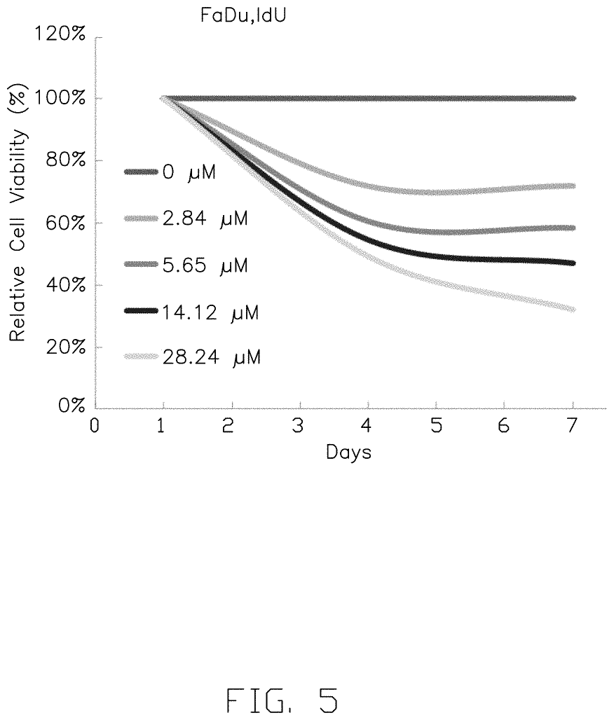

Referring to FIG. 5, an in vitro cell viability chart of FaDu cells when treated with IdU is provided in accordance with an exemplary embodiment of the present disclosure. FaDu cells are treated with different dosages of IdU for 24 hours and then FaDu cells are analyzed for cell viability. FaDu cells are seeded in plate and treated with IdU by similar experiment procedures with FIG. 4. FIG. 5 shows that the cell viability is around 50% when treated with 14.12 μM IdU on Day 7.

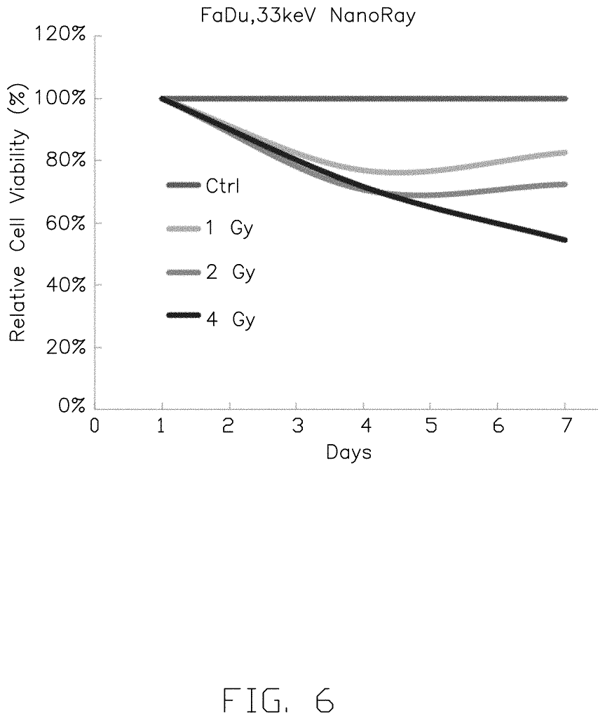

Referring to FIG. 6, an in vitro viability chart of FaDu cells when irradiated by NR is provided in accordance with an exemplary embodiment of the present disclosure. FaDu cells are treated with different radiation doses of NanoRay radiation (NR). FaDu cells are trypsinized and collected by centrifugation. The centrifuged FaDu cells are then re-suspended in PBS in a 1.5-ml tube with 5×10⁴ cells in 20 μL. The tube containing centrifuged FaDu cells are then subjected to different radiation doses of NR. FIG. 6 shows the cell viability is around 50% when applied with 4 Gy, 33 keV NR on Day 7. From FIGS. 4, 5 and 6, it is concluded that an optimized dosage for BrdU and IdU for in vivo experiments would be 10 μM, and the optimized radiation dose for NR is within 4 Gy.

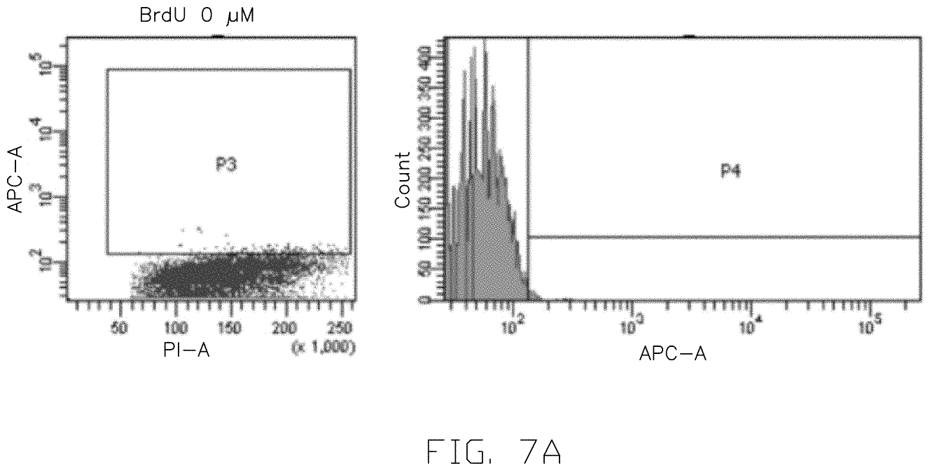

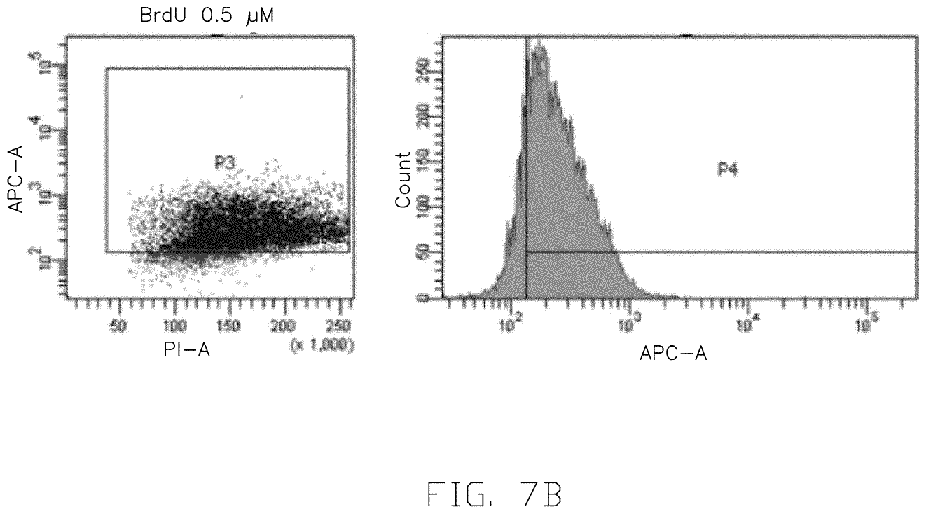

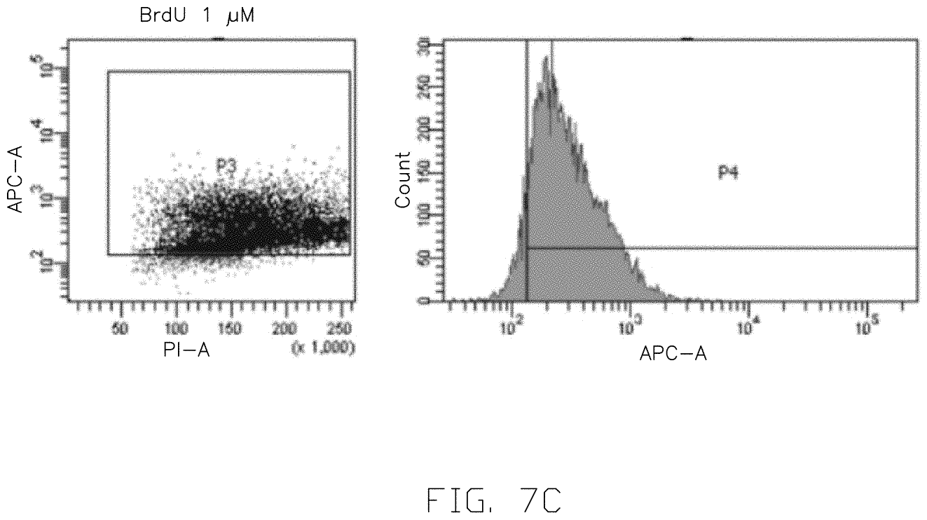

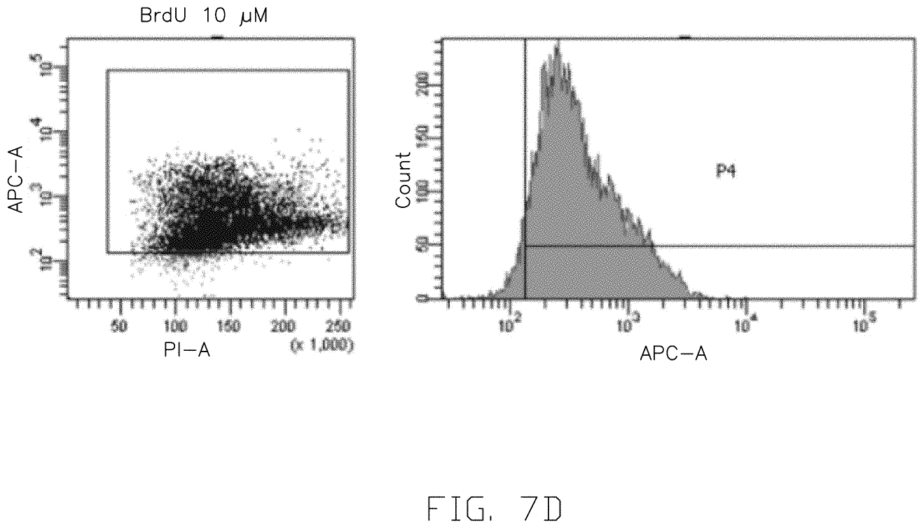

Referring to FIG. 7A, a flow cytometry analysis of FaDu cells without BrdU treatment is provided in accordance with an exemplary embodiment of the present disclosure. FaDu cells are analyzed by flow cytometry using BrdU Flow Kits (BD Pharmingen), in absence of BrdU. FaDu cells are trypsinized, washed, fixed, permeablized, blocked, and then treated with DNase I (5 U/10⁶ cells/100 μL) for 1 hour at 37° C. FaDu cells are then hybridized with anti-BrdU antibodies for 1 hour at room temperature. After several washes, FaDu cells are analyzed for fluorescent signals with LSR II Analytic Flow Cytometer (BD Biosciences). FIG. 7A shows almost no fluorescent signals are detected when no BrdU is presented, only less than 1% of FaDu cells are detected with the fluorescent signals.

Referring to FIGS. 7B-D, flow cytometry analyses of FaDu cells when treated with BrdU are provided in accordance with an exemplary embodiment of the present disclosure. In FIGS. 7B-D, FaDu cells are analyzed by flow cytometry with the same experiment procedures for FIG. 7A. FaDu cells in FIGS. 7B, 7C, and 7D are respectively treated with 0.5 μM BrdU, 1 μM BrdU, and 10 μM BrdU for 24 hours. FIG. 7B shows that when treated with 0.5 μM BrdU, about 83% of FaDu cells can be labeled. FIGS. 7C and 7D show that when FaDu cells are treated with more than 1 μM of BrdU, more than 90% of FaDu cells can be labeled. The DNA incorporation efficiency of BrdU is 1 μM to more than 90% of FaDu cells, therefore 1 μM would be used in the subsequent experiment as an optimized dosage of BrdU.

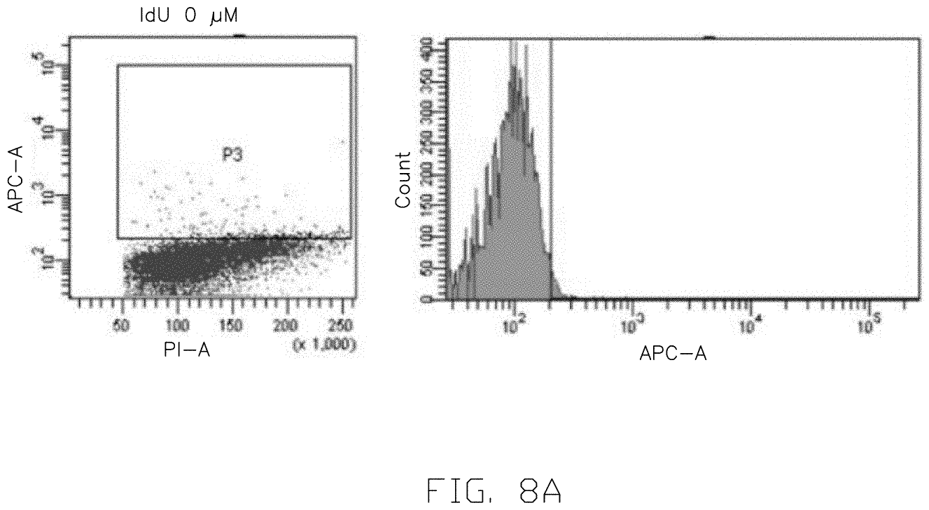

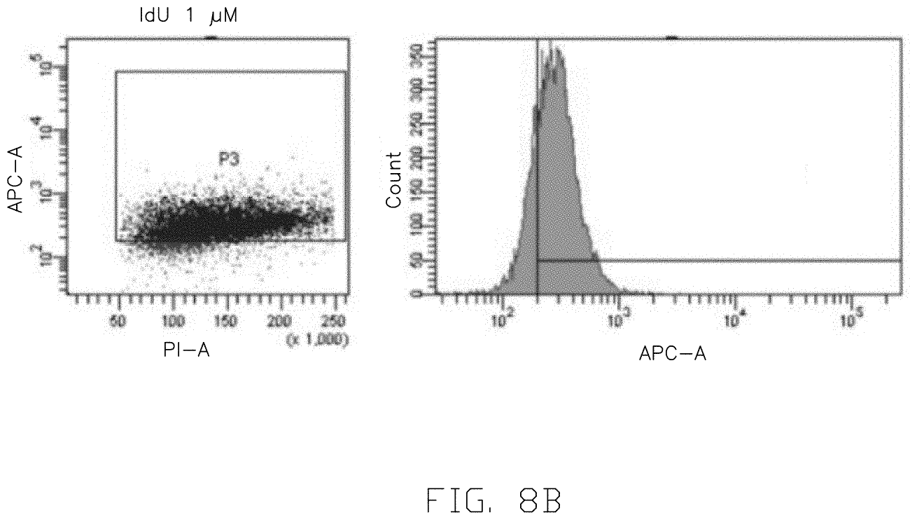

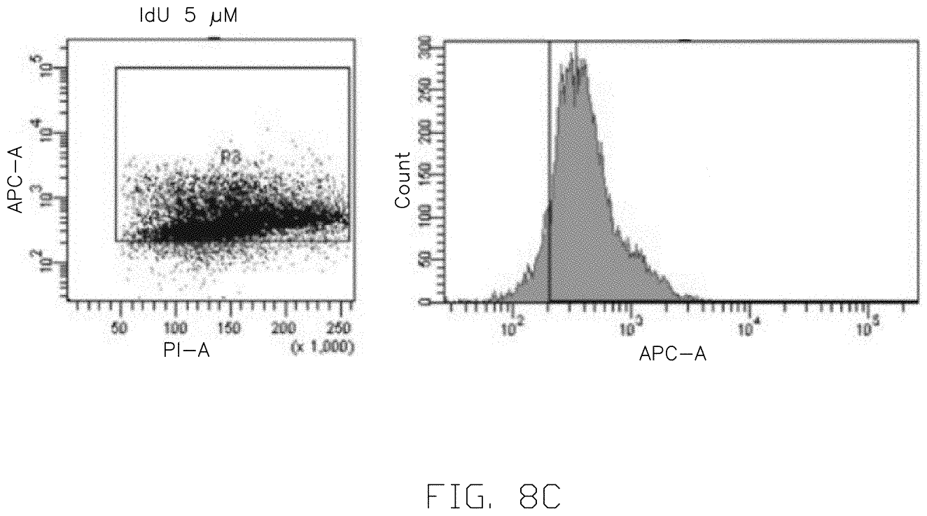

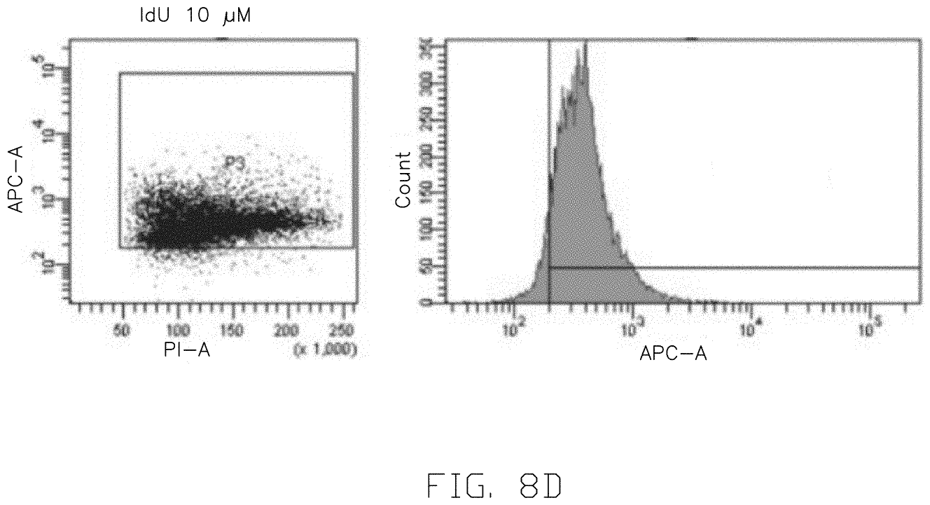

Referring to FIG. 8A, a flow cytometry analysis of FaDu cells without IdU treatment is provided in accordance with an exemplary embodiment of the present disclosure. FaDu cells are analyzed by flow cytometry using BrdU Flow Kits (BD Pharmingen), in absence of IdU. FaDu cells are treated with similar experiment procedures as in FIG. 7A, but anti-BrdU antibodies are replaced with anti-IdU antibodies. For the detection of IdU-incorporated cells, FaDu cells being treated by anti-IdU antibodies are then hybridized with a secondary antibody conjugated by proper fluorescent dye for 1 hour at room temperature. FIG. 8A shows almost no fluorescent signals are detected when no IdU is presented only less than 1% of FaDu cells are detected with the fluorescent signals.

Referring to FIGS. 8B-D, flow cytometry analyses of FaDu cells when treated with IdU are provided in accordance with an exemplary embodiment of the present disclosure. FaDu cells are analyzed by flow cytometry with similar experiment procedures as in 7A. However, FaDu cells in FIGS. 8B, 8C, and 8D are treated with 1 μM IdU, 5 μM IdU, and 10 μM IdU for 24 hours, respectively. FIG. 8B shows that when treated with 0.5 μM, about 84% of FaDu cells can be labeled. FIGS. 8C and 8D show that when FaDu cells are treated with more than 10 μM of IdU, more than 90% of FaDu cells can be labeled. The DNA incorporation efficiency of IdU is 10 μM to more than 90% of FaDu cells, therefore 10 μM would be used in the subsequent experiment as an optimized dosage of IdU.

Referring to FIGS. 9A-C, an in vitro cell viability chart of FaDu cells when treated with BrdU alone, NR alone, and BrdU and NR, is provided in accordance with an exemplary embodiment of the present disclosure. NR treatments in FIGS. 9A, 9B, and 9C are similar with the experiment procedures for FIG. 6. In samples where FaDu cells are treated with BrdU and NR, FaDu cells are incubated with BrdU for 24 hours before irradiated with NR. Referring to FIG. 9B, a colony forming assay result of FaDu cells after the treatments described in FIG. 9C, is provided in accordance with an exemplary embodiment of the present disclosure. The colony forming assay in FIG. 9B are proceeded as following: FaDu cells treated by different regimens are grouped and seeded in 6-well-plates with 1000 cells in each of the well and cultured for 14 days; the culture medium was replaced every 3 to 4 days; in the end of the experiment, the cells are washed twice with PBS and fixed with 4% paraformaldehyde for 15 minutes and stained with 0.1% crystal violet. Referring to FIG. 9C, a quantitative result of the colony forming assay in FIG. 9B is provided in accordance with an exemplary embodiment of the present disclosure. In FIG. 9C, the results of the colony forming assay are quantified by microscopic examination by stereomicroscope.

FIGS. 9A, 9B, and 9C show that when 1 μM BrdU is combined with 1 Gy of 14 keV NR, the cell viability of FaDu cells is 46.8% on Day 7, and the colony forming inhibition of FaDu cells is 57.5%. The colony forming inhibition is related to the colony formation efficiency in FIG. 9C, wherein the colony forming inhibition (%) plus the colony forming efficiency (%) is 100%. A higher colony forming inhibition indicates a lower colony forming efficiency. Because BrdU or NR has less cytotoxicity to FaDu cells when used alone in FIGS. 4 and 6, the combination of BrdU and NR shows strong synergistic cytotoxicity to FaDu cells, and has a range of effective dosages of 0.5-2 μM BrdU and 0.1-1 Gy of 14 keV NR. FIG. 9A also shows that the combination of BrdU and NR is much more effective on FaDu cells than BrdU or NR used alone. The combination of BrdU and NR provided by the present disclosure has demonstrated synergistic cytotoxicity to cancer cells, and can be a viable therapeutic cancer treatment in vivo.

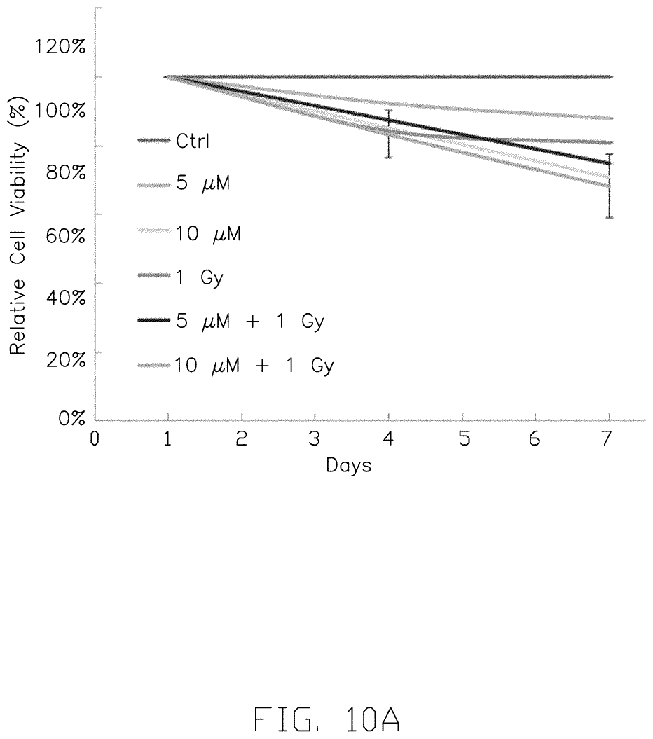

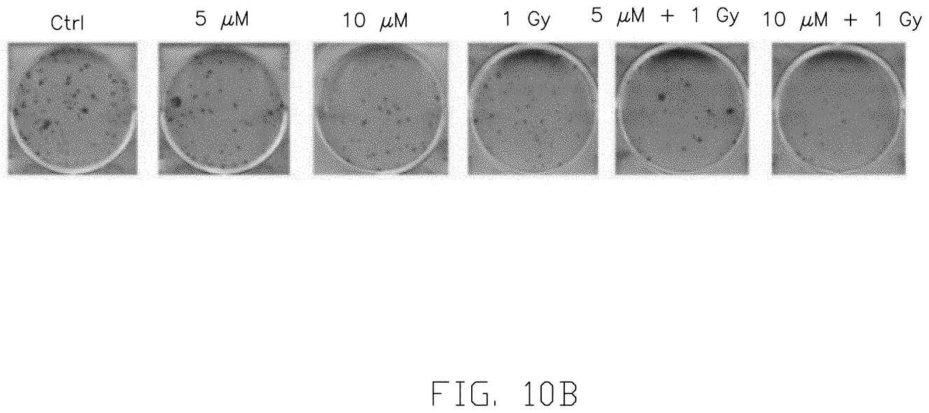

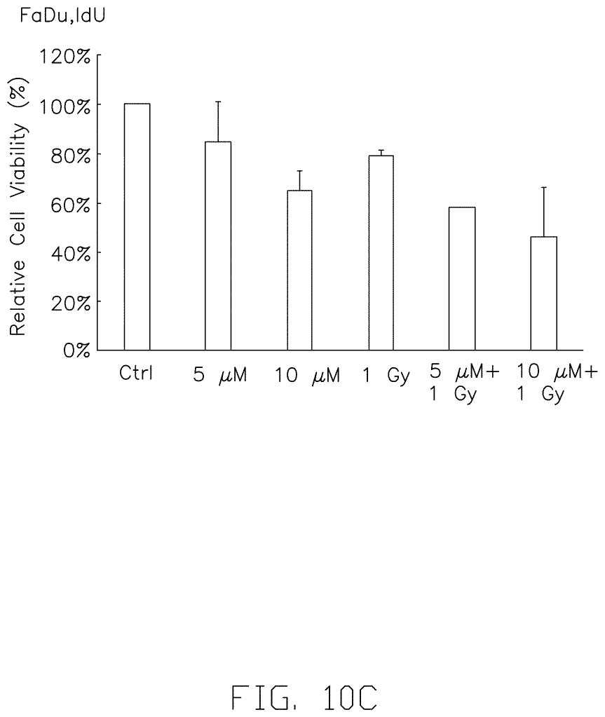

Referring to FIGS. 10A and 10B, an in vitro cell viability chart and a colony forming assay result of FaDu cells are provided in accordance with an exemplary embodiment of the present disclosure. FIG. 10C is a quantitative result of FIG. 10B, in accordance with an exemplary embodiment of the present disclosure. In FIGS. 10A-C, FaDu cells are treated with IdU, NR, and a combination of IdU and NR. NR treatments in FIGS. 10A, 10B, and 10C are similar to the experiment procedures for FIG. 6. In samples where FaDu cells are treated with IdU and NR, FaDu cells are incubated with IdU for 24 hours before irradiated with NR. The colony forming assay shown in FIGS. 10B and 10C are of similar experiment procedures as in FIGS. 9B and 9C. Because the iodine atom in IdU has a higher k-edge, radiation energy of NR has been adjusted to 33 keV. FIGS. 10A, 10B, and 10C show that when 10 μM IdU is combined with 1 Gy of 33 keV NR, the cell viability of FaDu cells is lower and the colony forming inhibition of FaDu cells is higher, than using IdU or NR alone.

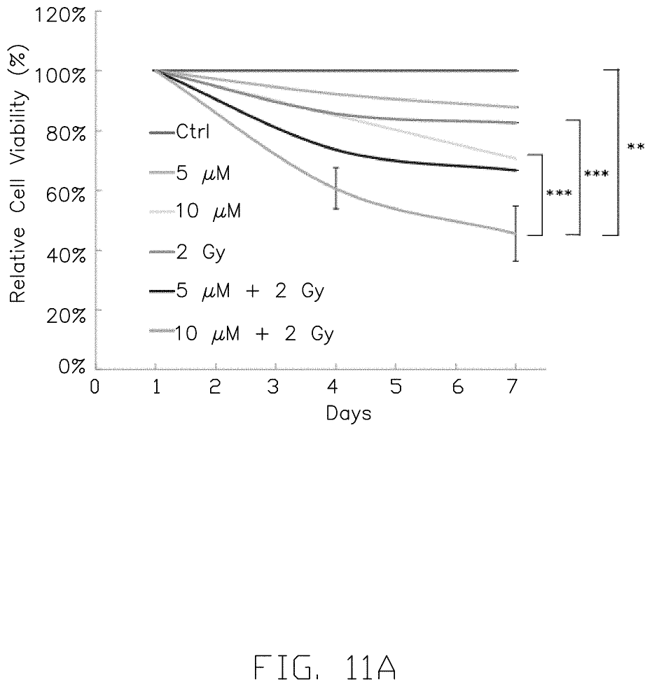

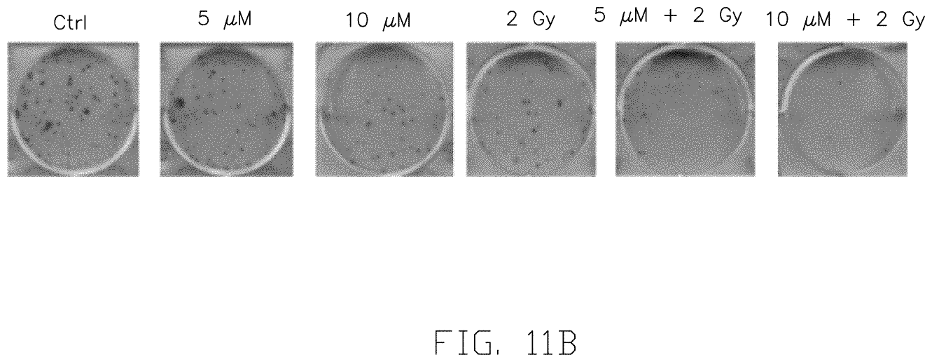

Referring to FIGS. 11A, 11B, and 11C, FaDu cells are treated with IdU, NR, and a combination of IdU and NR. The radiation dose of NR is adjusted to 2 Gy. The colony forming assay shown in FIGS. 11B and 11C are similar to the experiment procedures for FIG. 9B. FIGS. 11A, 11B, and 11C show that when 10 μM IdU is combined with 2 Gy of 33 keV NR, the cytotoxicity to FaDu cells is significantly better than those treated by IdU or NR alone.

The combination of IdU and NR shows strong synergistic cytotoxicity to FaDu cells, and has a range of effective dosages of 5-20 μM IdU and 0.1-2 Gy of 33 keV NR. The combination of IdU and NR provided by the present disclosure has demonstrated a significant cytotoxicity to cancer cells, and can be a viable therapeutic cancer treatment in vivo.

### 1.2 Combining IdU and Monochromatic X-Ray In Vivo

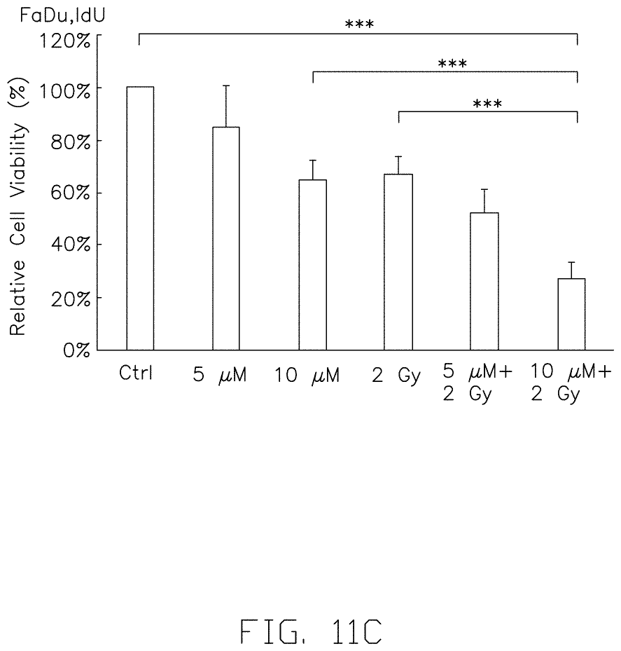

The in vivo cytotoxicity to cancer cells from the combination of iododeoxyuridine (IdU) and monochromatic X-ray are demonstrated. FIG. 12 is a schematic diagram of an in vivo animal experiment, in accordance with an exemplary embodiment of the present disclosure. A subcutaneous malignant solid tumor comprised of FaDu cells (tumor size is about 200 mm³) on the tumorigenic mice is injected with 0.5 μg/μl of IdU. 4 hours after the injection, the solid tumor is then irradiated by 4 Gy 33 keV NR. The IdU-NR treatment are repeated on the same tumor after 7 days. Other groups of mouse are only injected with 50 μg of IdU or only irradiated with 4 Gy 33 keV NR, and the IdU injection or the NR irradiation is repeated on the same tumor after 7 days. The control group is the mice without NR, IdU, or IdU-NR treatment.

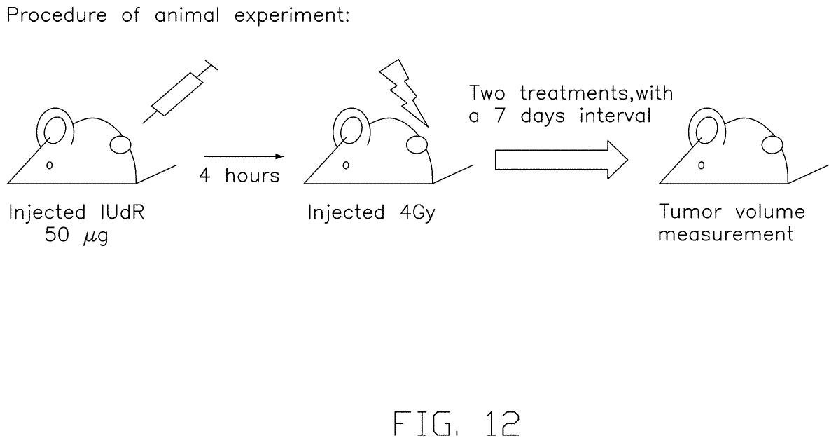

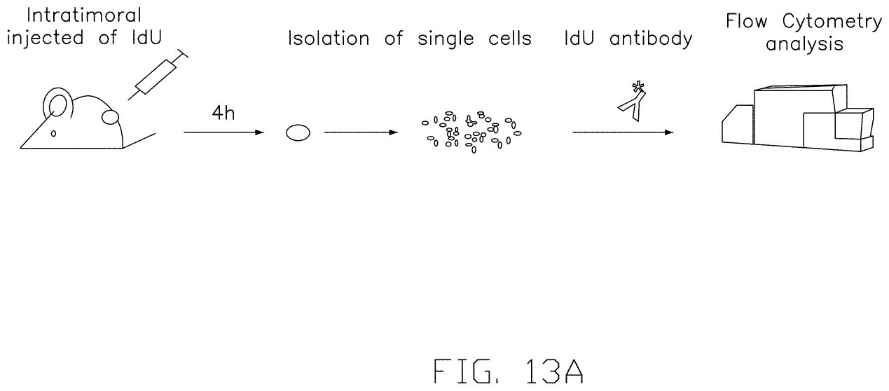

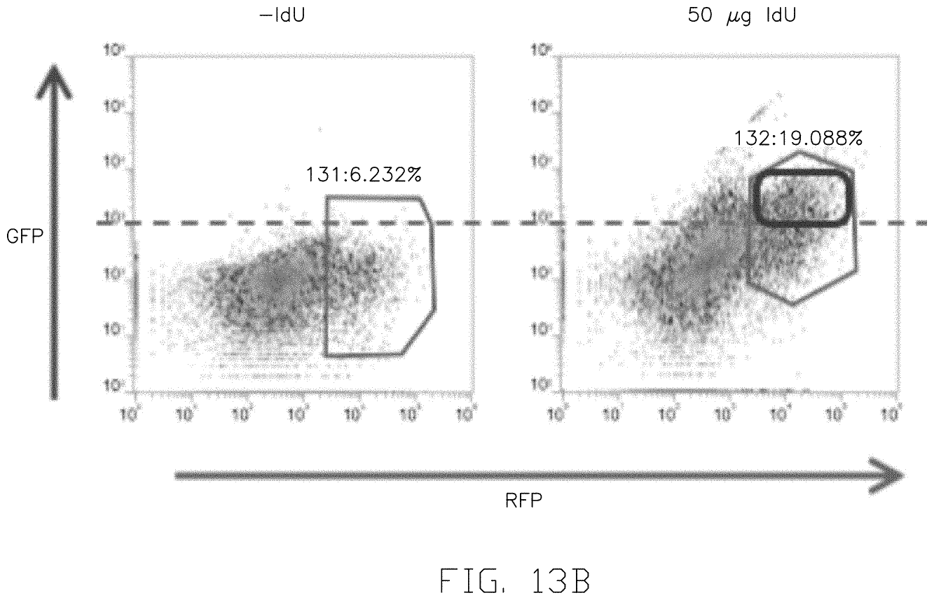

An injection concentration of IdU is selected based on a flow cytometry analysis on the incorporation rate of IdU in tumor cells. Referring to FIG. 13A, a schematic diagram of an in vivo animal experiment for determining an injection concentration of IdU is provided in accordance with an exemplary embodiment of the present disclosure. FaDu cells are marked with RFP (red fluorescent protein) and IdU is marked with GFP (green fluorescent protein). The malignant solid tumors formed by FaDu-RFP+ cells on the tumorigenic mouse are injected with different concentrations of IdU: 100 μg, 50 μg, 20 μg, 10 μg, 5 μg, or 2 μg per 100 μl PBS. 4 hours later, the tumorigenic mouse are sacrificed and the solid tumors are isolated to single cells. The single cells from the solid tumors are then subjected to antibody staining and flow cytometry analyses. Referring to FIG. 13B, a flow cytometry result of the experiments described by FIG. 13A, is provided in accordance with an exemplary embodiment of the present disclosure. The horizontal axis represents the RFP signals, and the vertical axis represents the GFP signals. The IdU incorporation in the single cells is indicated by cells with IdU+/GFP+. The left panel of FIG. 13B shows FaDu cells without IdU treatment, an area 131 is FaDu cells detected with GFP signals. Only 6.232% of FaDu cells are detected with GFP in the left panel of FIG. 13B. The right panel of FIG. 13B are FaDu cells treated with 50 μg of IdU, an area 132 is FaDu cells detected with GFP signals. 19.088% of FaDu cells are detected with GFP signals in the right panel of FIG. 13B. Referring to FIG. 13C, a IdU incorporation rate bar chart is provided in accordance with an exemplary embodiment of the present disclosure. Percentages of IdU+/GFP+ cells in the FaDu-RFP cells injected with different IdU concentration are shown, and the cells injected with 50 μg IdU/100 μl PBS have the best IdU incorporation rate among other concentrations. Therefore, 50 μg IdU/100 μl PBS is an optimal injection concentration for the IdU in vivo tests of the present disclosure.

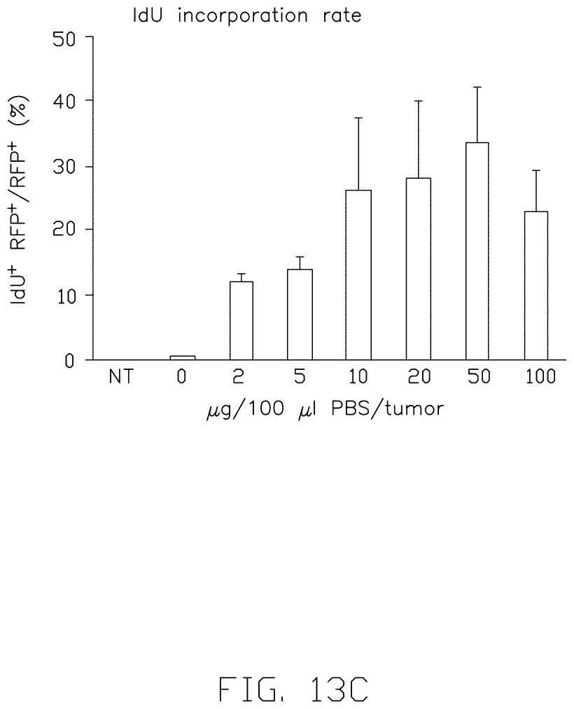

FIG. 14 shows the results of a tumor growth chart in an in vivo animal experiment, in accordance with an exemplary embodiment of the present disclosure. The subcutaneous malignant solid tumor comprised of FaDu cells on the tumorigenic mice is treated with IdU, NR, or IdU and NR, following the same experiment procedure as depicted in FIG. 12. The relative proliferation rates of the solid tumors are calculated by measuring the size of the solid tumor on Days 3, 7, 10, 14, 17, 21, 24, and 28, respectively. The solid tumor in the control group has a highest relative proliferation rate of exceeding 1400%, whereby the solid tumors of IdU-only and NR-only groups has lower relative proliferation rate than the control group. The solid tumor of IdU-NR group has the lowest relative proliferation rate among other groups, and the relative proliferation rate of IdU-NR group is even lower than 100% (p<0.05), this indicates the tumor size is smaller after the IdU and NR treatment. The combination of IdU and NR provided by the present disclosure has demonstrated synergistic cytotoxicity to cancer cells in the tumorigenic mouse model.

Although the results in FIGS. 9A-9C suggest that the combination of BrdU and 14 keV NR generates a pronounced synergistic anti-cancer effect than the combination of IdU and 33 keV NR in vitro, 14 keV NR has a half-value layer (HVL) of only about 0.35 cm in soft-tissue (not shown). The half-value layer is a thickness of any given material wherein 50% of an incident energy is attenuated at the thickness, and is inversely proportional to an attenuation coefficient. Therefore, the combination of IdU and 33 keV NR of the present disclosure is used in the following in vivo experiment.

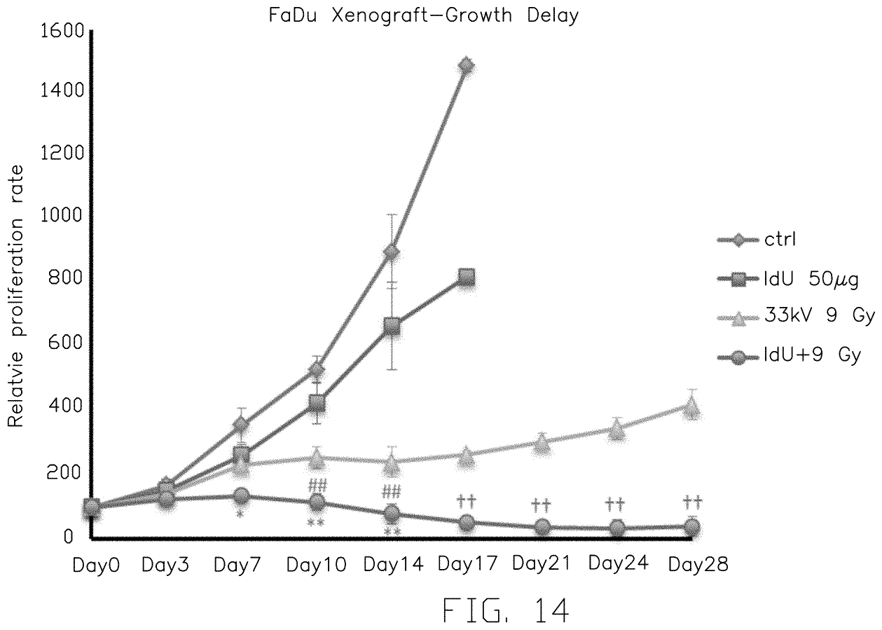

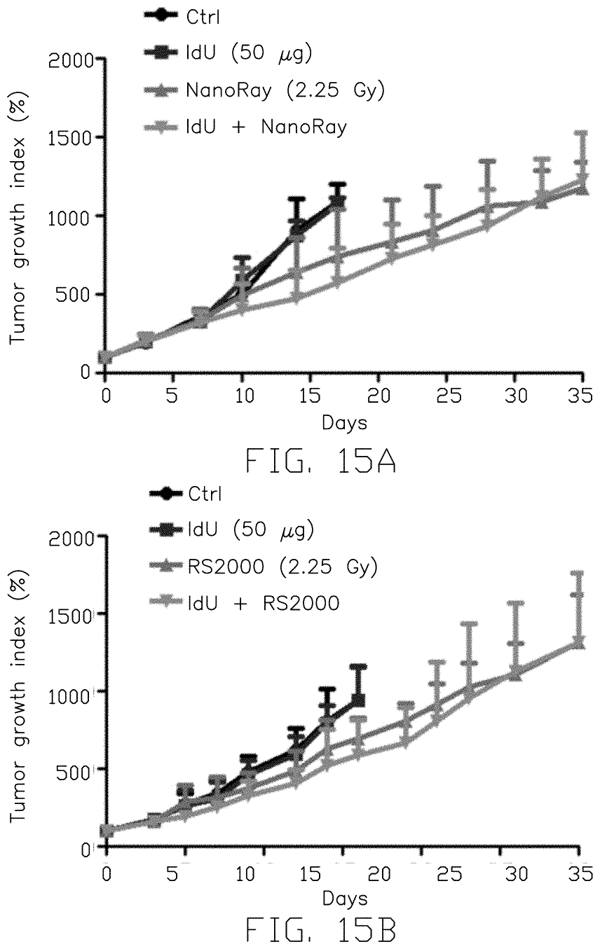

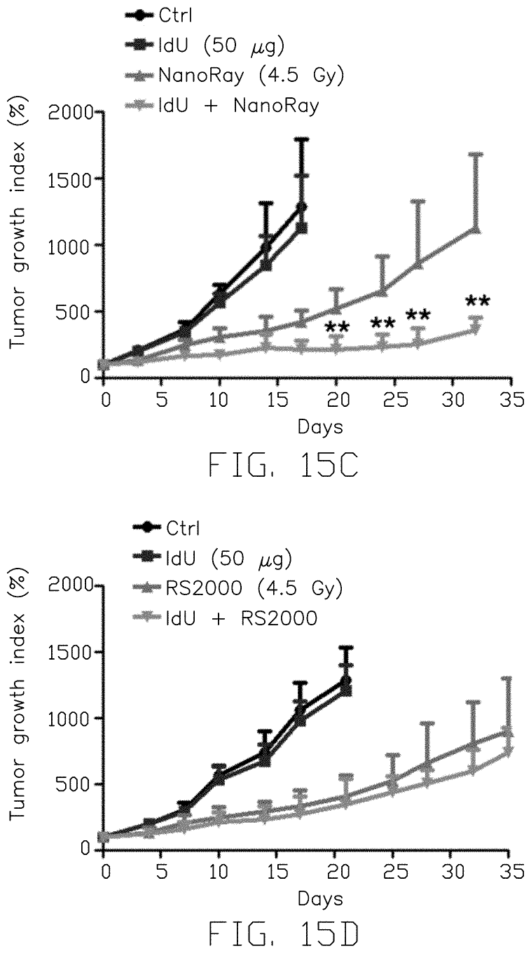

To further investigate a therapeutically effective dosage for the combination of IdU and NR, another series of in vivo experiments are conducted, see FIGS. 15A-15F. The experiment procedure is the same with the procedure described in FIG. 12. FIG. 15A and FIG. 15B show cells irradiated with 2.25 Gy NR and 2.25 Gy RS2000, FIG. 15C and FIG. 15D show cells irradiated with 4.5 Gy NR and 4.5 Gy RS2000, and FIG. 15E and FIG. 15F show cells irradiated with 9 Gy NR and 9 Gy RS2000, in accordance with exemplary embodiments of the present disclosure. Control groups in FIGS. 15A-15F are FaDu cells isolated from the subcutaneous solid tumor of the tumorigenic mouse without treatment of IdU or any radiation. The tumor growth index in FIGS. 15A-15F are calculated based on tumor volumes: for each of the tumorigenic mouse, the tumor volume on Day 0 is set as 100%, and relative volume percentages in the following days relative to Day 0 are presented as the tumor growth index (%).

In FIGS. 15A-15F, cells treated only with IdU demonstrate similar results with the control group: the tumor growth is not inhibited. Cells irradiated by NR only demonstrate the tumor growth is inhibited in a dose-dependent manner. When cells are treated with the combination of IdU and 4.5 Gy or 9 Gy NR, the tumor growth are inhibited more significantly than the cells only irradiated by NR. In FIG. 15C, the combination of IdU and 4.5 Gy NR shows a tumor growth inhibition rate of 68% at the end of the in vivo test. In FIG. 15D, the combination of IdU and 9 Gy NR shows the tumor growth inhibition rate of 83% at the end of the in vivo test. In FIGS. 15B, 15D, and 15F, a combination of IdU and RS2000 does not significantly inhibit the tumor growth, when comparing with cells only irradiated by RS2000.

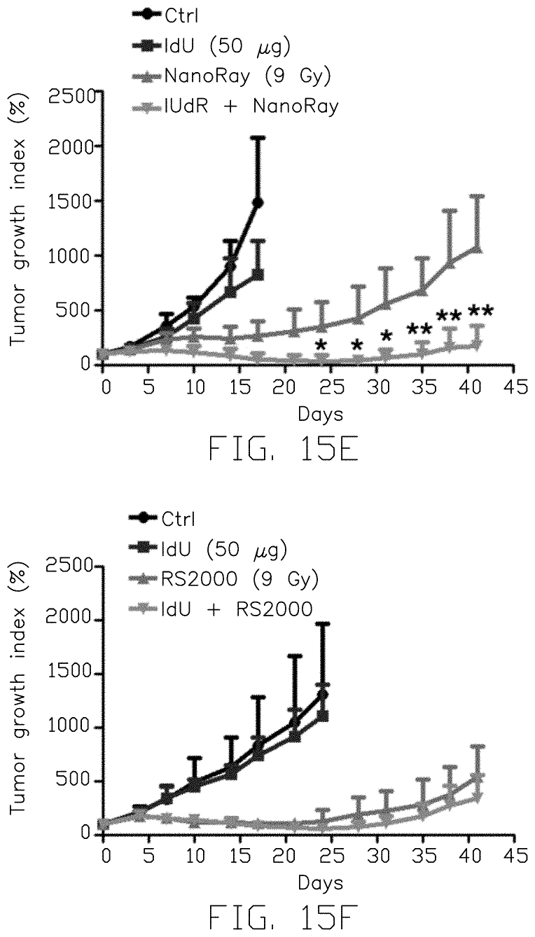

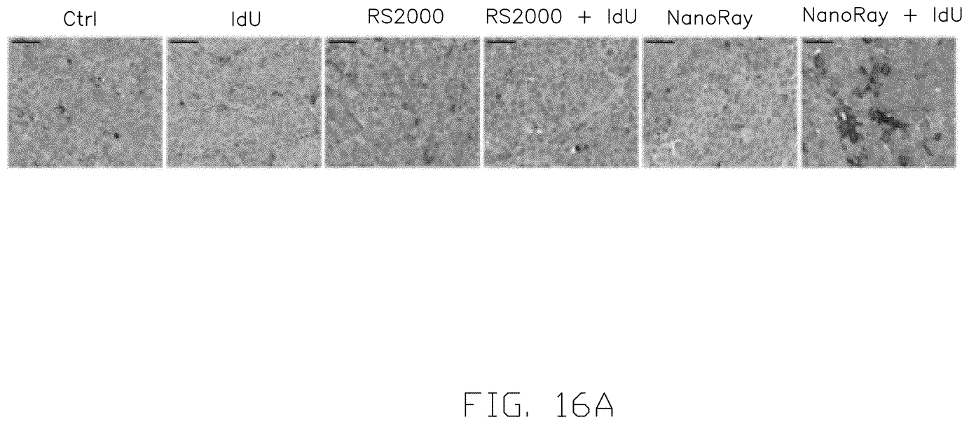

One day after the tumorigenic mouse are treated with IdU, or the combination of IdU and 4.5 Gy of NR/RS2000, the subcutaneous FaDu tumor cells are subjected to immunohistochemical (IHC) examination. Referring to FIGS. 16A and 16B, IHC results are demonstrated qualitatively and quantitatively, in accordance with an exemplary embodiment of the present disclosure. The control group in FIGS. 15A-15F or FIGS. 16A-16B is the subcutaneous FaDu tumor cells without any treatment. Referring to FIG. 16A, FaDu cells treated with the combination of IdU and 4.5 Gy of NR has more apoptotic cells than FaDu cells in other groups. Referring to FIG. 16B, the quantitative results of IHC examination show that, when comparing with other treatments, the combination of IdU and NR induces more pronounced CASPASE-3 expression in the tumor cells. CASPASE-3 is directly involved in apoptosis, therefore the combination of IdU and NR is proven to induce tumor growth inhibition.

The therapeutically effective dosage of IdU in a pharmaceutical composition for human in the combination of IdU and NR can be calculated from FIGS. 15A-15F. Considering the differences on metabolism, body weight, pharmacokinetics, and pharmacodynamics between mouse and human, an injection volume of IdU can be 4.5%-70% of the tumor volume in human, when injecting IdU to a solid tumor of a human subject. In the in vivo experiments of FIG. 15A-15F, the injection volume is 50% of the initial tumor volume, and the concentration of IdU is 50 μg/100 μl PBS. Considering the differences on metabolism, body weight, pharmacodynamics, and pharmacodynamics between mouse and human, a concentration of IdU being injected to the human subject could be 0.5 mg/ml. From a clinical perspective, recurrent head-and-neck tumor in human patients that are not suitable for surgical resection have a size range of 30-500 cm³, and the injection volume of other anti-tumor drugs when administered to the solid tumor is about 10% to 30% of the tumor volume. Therefore, the injection volume of IdU for the human patient ranges from 7.5 ml to 125 ml, according to the present disclosure. The injection volume for human patient is then multiplied by the concentration of IdU in FIGS. 15A-15F (0.5 mg/ml). Therefore, an injection amount of IdU for the human patient can be in a range of 3.75 mg to 62.5 mg. Therefore, the therapeutically effective dosage of IdU may fall within the range of injection volume of 7.5 ml to 125 ml, or the therapeutically effective dosage of IdU may fall within the range of injection amount of 3.75 mg to 62.5 mg.

To conclude, the therapeutically effective dosage of IdU in the pharmaceutical composition for the human patient in the Auger molecular therapy disclosed in the present disclosure comprises a minimum injection volume of 7.5 ml, a minimum injection amount of 3.75 mg, a maximum injection volume of 125 ml, or a maximum injection amount of 62.5 mg, according to the results in FIGS. 15A-15F and the head-and-neck tumor sizes in the human patients.

Additionally, the pharmaceutical composition comprising IdU may further comprise a pharmaceutical carrier that facilitates absorption and distribution of IdU in the human patient. The pharmaceutical carrier may be liposomes, nanofibers, protein conjugates, dendrimers, or microspheres (e.g., a biodegradable polymer poly (lactic-co-glycolic) acid microsphere).

To investigate the mechanism associated with the combination of IdU and NR in the Auger molecular therapy, molecular markers involving DNA damage and apoptosis are analyzed. In vitro FaDu cells are treated with 10 μM of IdU, and irradiated by 2Gy of NR or RS2000. 30 minutes after above treatments, FaDu cells are subjected to Western blot to determine the expression of several proteins relevant to DNA damage and apoptosis: phosphor-ATM (pATM) is a protein kinase activated by DNA double-strand-breaks (DSBs), and it phosphorylates several key factors to initiate DNA damage checkpoint and repair; γH2AX is one of the targets of pATM, and it forms the foundation of chromatin-based signaling cascades at DSBs sites, and it is widely considered as a highly specific and sensitive marker for quantitative evaluation of DSB; and CASP3 is a marker for increased apoptosis, wherein c-CASP3 is a cleaved CASP3 formed when apoptotic signals are present in the cell. The subcutaneous tumor also subjected to Western blot 3 days after the above treatments.

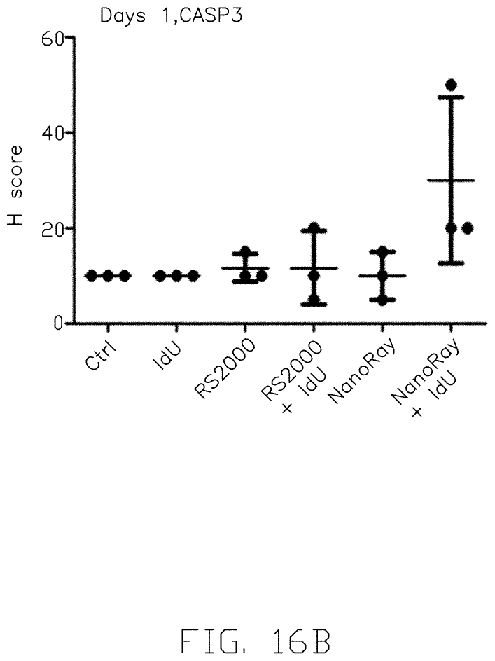

FIGS. 17A and 17B are results of Western blotting for protein expression levels of pATM, γH2AX, and c-CASP3, in accordance with exemplary embodiments of the present disclosure. FIG. 17A is the result at 30 minutes after irradiation, and FIG. 17B is the result at 3 days after irradiation, wherein β-ACTIN are served as an internal control in both figures. In FIG. 17A, the expression of pATM and γH2AX proteins are evident in the cells treated with RS2000, RS2000 and IdU, NR, and NR and IdU. Specifically, the combination of NR and IdU induces the most γH2AX expression among all other treatments. Referring to FIG. 16B, the expression of CASP3 are increased.

The results in FIG. 14 demonstrate that the combination of IdU and NR has synergistic cytotoxicity to the cancer cells in the tumorigenic mouse model. The results in FIGS. 15A-15F demonstrate that the therapeutic dosage in the tumorigenic mouse model for the combination of IdU and NR can be 9 Gy or 4.5 Gy of NR and 0.5 μg/μl of IdU, and that the therapeutically effective dosage for IdU in the pharmaceutical composition of the Auger molecular therapy of the present disclosure may comprise the injection volume of 7.5 ml-125 ml, or the therapeutically effective dosage of IdU may comprise the injection amount of 3.75 mg-62.5 mg. The results in FIGS. 16A-16B, 17A-17B demonstrate the mechanism associated with the combination of IdU and NR in the Auger molecular therapy involves DNA damage and apoptosis of the cancer cells.

2. Combining Dibromocurcumin (DBC) with Monochromatic X-Ray in Auger Molecular Therapy

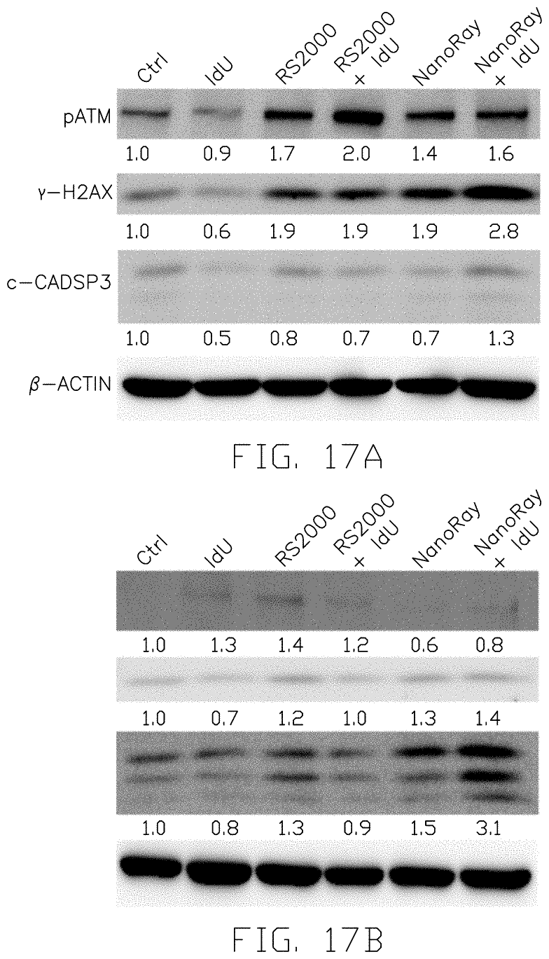

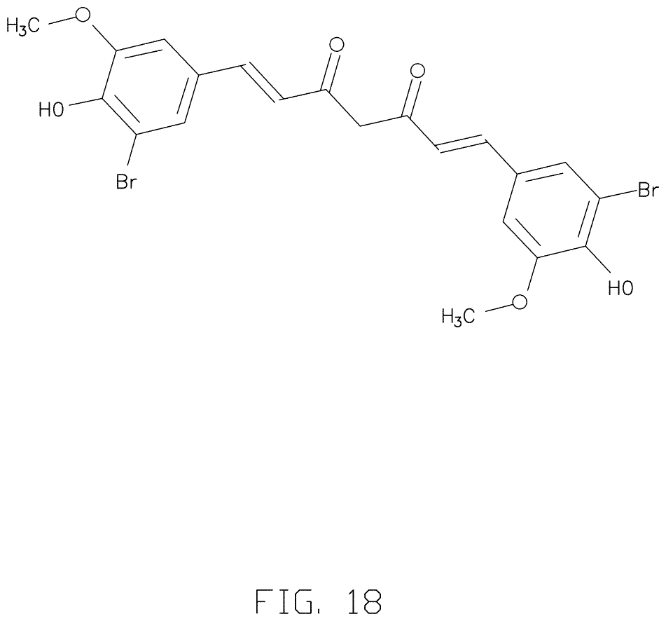

Dibromocurcumin (DBC; CAS number: 869789-04-8) is a diarylheptanoid of curcuminoids, which are natural phenols. The curcuminoids has been found to have anti-inflammation and anti-oxidation effects, and could be an inhibitor on cancer cell growth via signal transduction. Referring to FIG. 18, a chemical structure of DBC is provided in accordance with an exemplary embodiment of the present disclosure. The curcuminoid molecule has two bromide conjugates on benzene groups to form the DBC of the present disclosure. Therefore, it is an object of the present disclosure to combine the bromide conjugation on DBC with monochromatic X-ray for synergistic cytotoxicity to cancer cells.

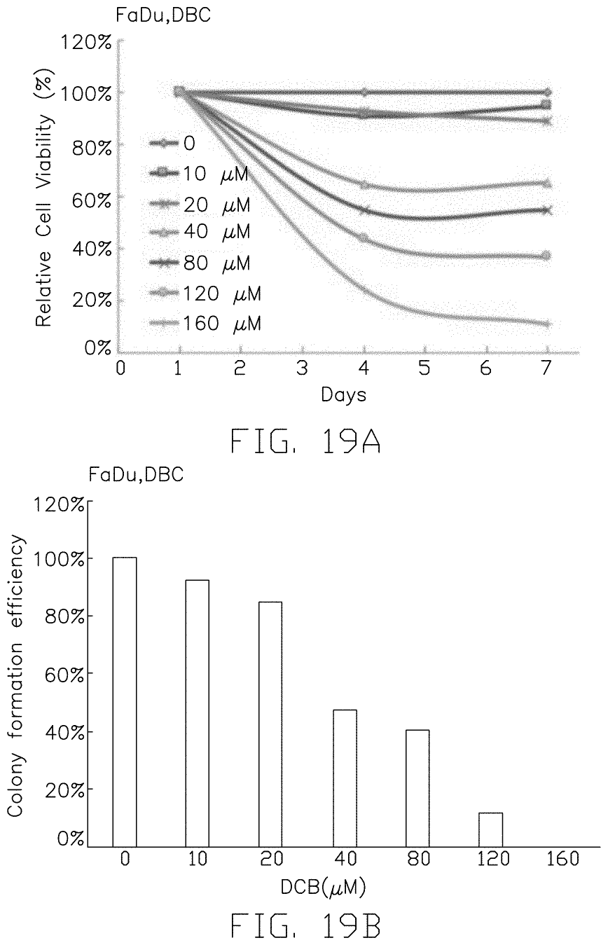

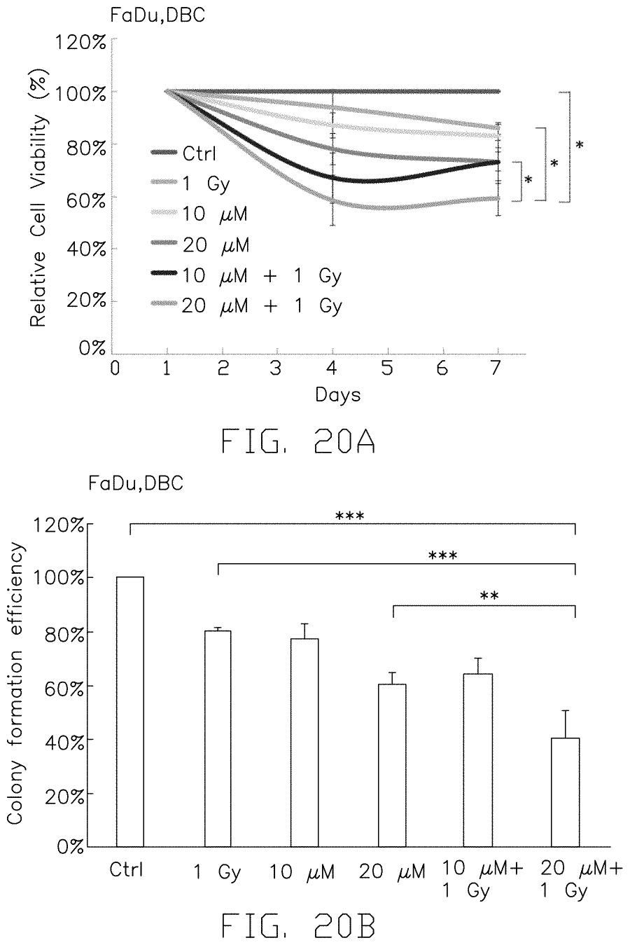

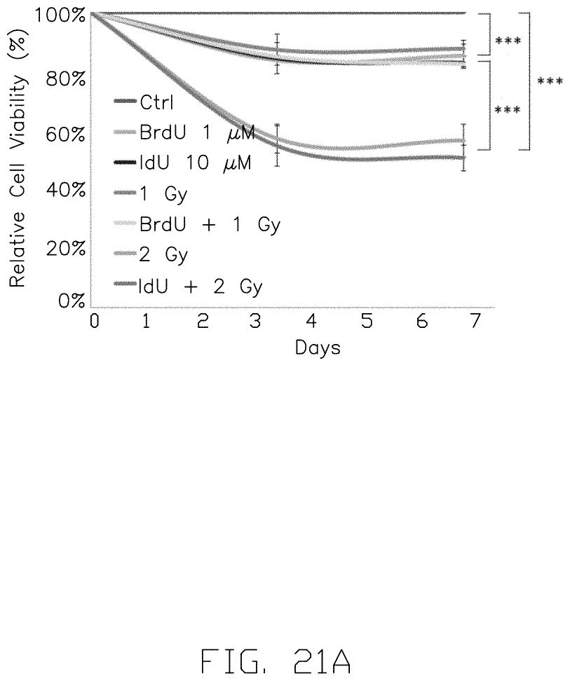

Referring to FIGS. 19A and 19B, an in vitro cell viability chart and a quantitative result for a colony forming assay are provided in accordance with an exemplary embodiment of the present disclosure. In FIGS. 19A and 19B, FaDu cells are treated with different dosages of DBC to determine an optimized dosage of DBC for conducting the Auger molecular therapy. Referring to FIGS. 20A and 20B, another in vitro cell viability chart and another quantitative result for a colony forming assay are provided in accordance with an exemplary embodiment of the present disclosure. In FIGS. 20A and 20B, FaDu cells are treated with different dosages of DBC and 1 Gy NanoRay radiation (NR) to determine an optimized dosage of DBC and NR. The control groups (Ctrl) in FIGS. 19A-19B, 20A-20B are cells without DBC and NR treatments.

Referring to FIG. 19A, FaDu cells are treated with different dosages of DBC and then FaDu cells are analyzed daily for cell viability. FaDu cells are treated with 0 μM DBC, 40 μM DBC, 80 μM DBC, 120 μM DBC, and 160 μM DBC for 24 hours, respectively. FIG. 19A shows that cell viability is less than 50% when treated with 80 μM DBC on Day 7.

Referring to FIG. 19B, FaDu cells are treated with different dosages of DBC and colony forming assays similar to that of FIG. 9B. FaDu cells are treated with 0 μM DBC, 40 μM DBC, 80 μM DBC, 120 μM DBC, and 160 μM DBC for 24 hours, respectively. FIG. 19B shows that in colony forming assay, the colony forming inhibition is about 50% when FaDu cells are treated with 40 μM DBC. FIGS. 19A and 19B also show DBC efficiently kills FaDu cells when the dosage is more than 120 μM, and demonstrate little cytotoxicity when the dosage is less than 20 μM.

Referring to FIG. 20A, FaDu cells are treated with different radiation doses of NanoRay radiation (NR) and different dosages of DBC. FaDu cells are incubated with DBC of 0 μM, 10 μM, or 20 μM for 24 hours, and then are subjected to similar experiment procedures as in FIG. 6. FIG. 20A shows when 20 μM of DBC is combined with 1 Gy of NR, the cytotoxicity to FaDu cells is significantly higher than using DBC or NR alone. Colony forming assays of FaDu cells in FIG. 20B shows the same result. The experiment procedure in FIG. 20B is similar to that of FIG. 9B. The combination of DBC and NR has synergistic cytotoxicity to FaDu cells, and has a range of effective dosages of 10-40 μM DBC and 0.1-1 Gy NR.

3. Combining BrdU and IdU with Conventional X-Ray Irradiation in Auger Molecular Therapy

The conventional X-ray irradiation cannot induce the Auger effects provided by the present disclosure. The conventional X-ray used in the following examples is RS2000 (Rad Source Technologies, Inc., GA, USA). RS2000 is a non-isotope irradiator that generates X-ray for biological research. RS2000 used in the following examples is set at 160 kVp and 25 mA. The spectrum of RS2000 is provided in FIG. 3.

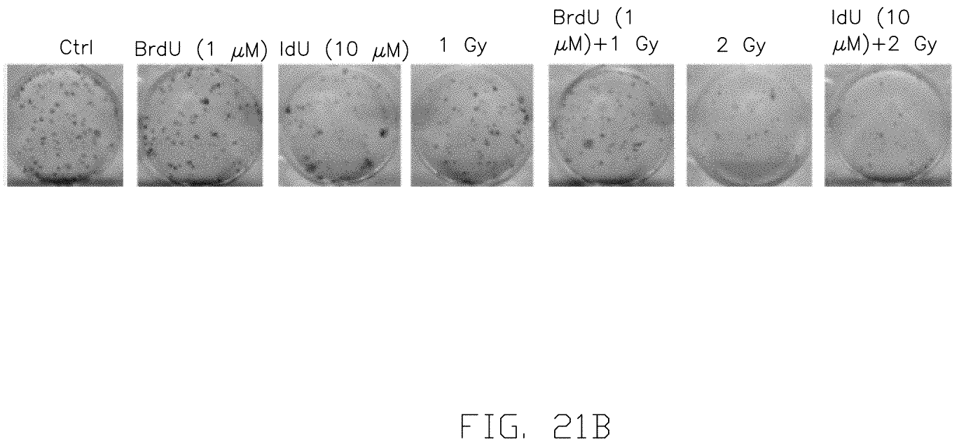

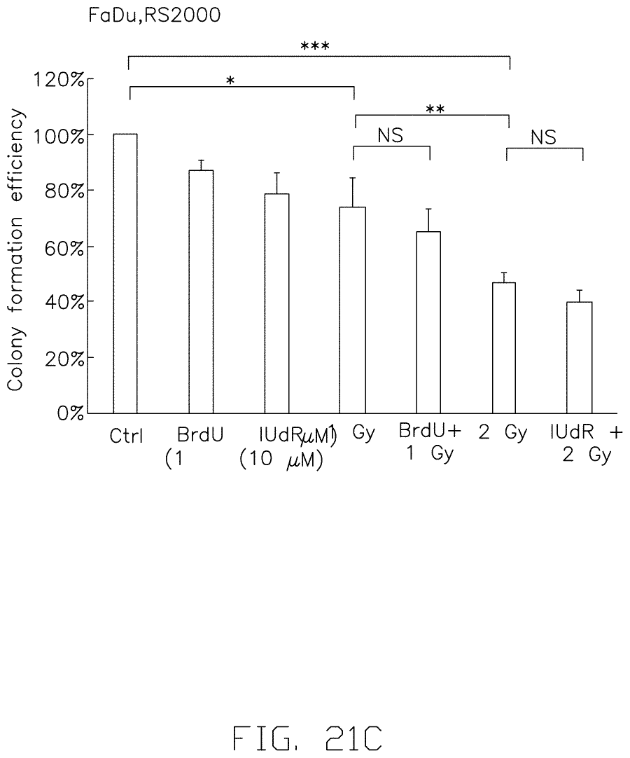

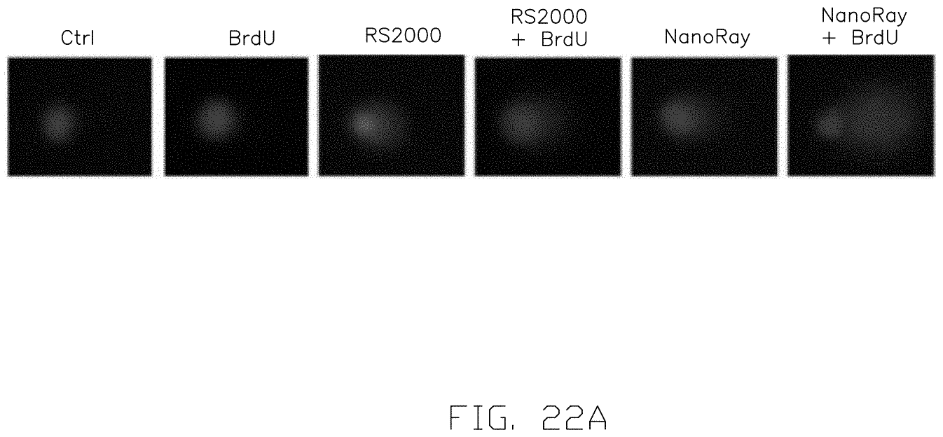

Referring to FIGS. 21A and 21B, an in vitro cell viability chart and a colony forming assay result of FaDu cells are provided in accordance with an exemplary embodiment of the present disclosure. FIG. 21C is a quantitative result of FIG. 21B, in accordance with an exemplary embodiment of the present disclosure. In FIGS. 21A-21C, FaDu cells are treated with different doses of RS2000, or different dosages of BrdU or IdU to evaluate cell viability. The control groups (Ctrl) in FIGS. 21A-21C are cells without BrdU, IdU, and RS2000 treatments.

Referring to FIG. 21A, FaDu cells are incubated with 1 μM BrdU or 10 μM IdU for 24 hours. Some groups of FaDu cells are irradiated with 1 Gy RS2000 after treated with 1 μM BrdU, or 2 Gy RS2000 after treated 10 μM IdU. FIG. 21A shows that the group treated with 1 μM BrdU and 1Gy of RS2000 has a lower cell viability. Similar results are presented in the group treated with 10 μM IdU and 2Gy of RS2000. To conclude, the combination of BrdU or IdU with RS2000 has cytotoxicity to FaDu cells, but does not have synergistic effects when comparing with the group using RS2000 alone. Colony forming assays of FaDu cells in FIGS. 21B and 21C show similar results. The experiment procedure in FIGS. 21B and 21C are similar to that of FIGS. 9B and 9C.

### 4. Comparison Between Monochromatic X-Ray and Conventional X-Ray in Auger Molecular Therapy

In previous figures, Nanoray radiation (NR) is utilized as a source of monochromatic X-ray, and RS2000 is utilized as a source of conventional X-ray. Comet assays and p-γH2AX immunofluorescent staining are utilized to evaluate DNA damages and to compare the cytotoxicity of FaDu cells when using different electromagnetic radiation sources.

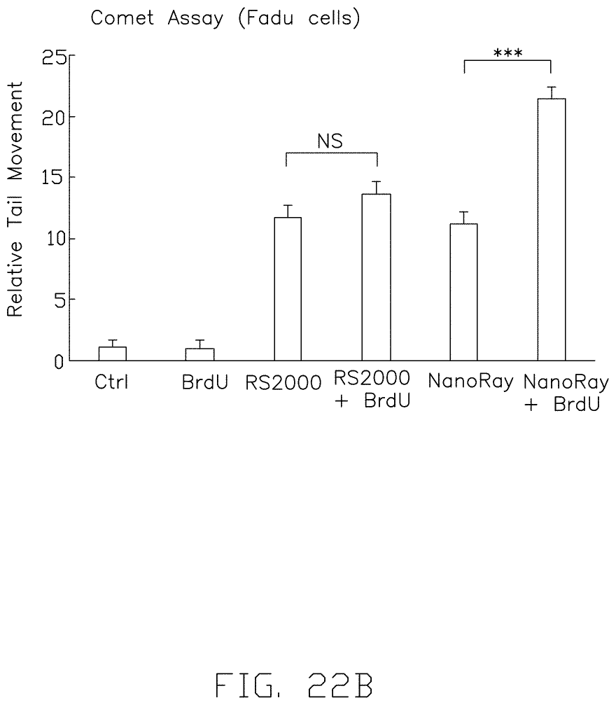

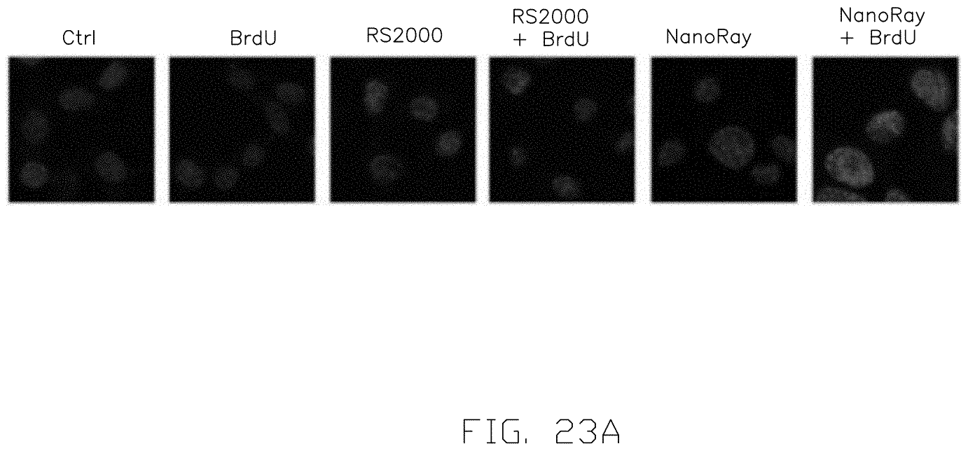

Referring to FIGS. 22A and 22B, comet assays for evaluating DNA damages induced by BrdU, RS2000, BrdU with RS2000, NR, and NR with BrdU are provided in accordance with an exemplary embodiment of the present disclosure. Comet assays are commonly used to measure DNA single-strand breaks (SSDs) and double-strand breaks (DSBs). FaDu cells (10⁴ cells/10 μl of PBS) are mixed with 75 μl of 0.75% low-melting agarose. The mixture is then immediately embedded onto a glass slide pre-coated with 2 layers of 1% agarose, and spread evenly with a coverslip. The mixture on the glass slide is solidified on ice for 5 minutes. 2 additional layers of 1% agarose are then coated on the cell layer. The cell is then soaked with cell lysis buffer (10 mM Tris, 2.5 M NaCl, 100 mM Na₂-EDTA, and 1% Triton X-100, pH 10.0) at 4° C. for 1 hour. The slide is then placed horizontally in an electrophoretic apparatus with alkaline electrophoretic buffer (300 mM NaCl and 1 mM Na₂-EDTA, pH >12.0) for 60 minutes, and then electrophoresis is conducted in the apparatus for 30 minutes at 25 V and 300 mA. After the electrophoresis has ended, the slide is soaked with neutralization buffer (0.4 M Tris, pH 7.4) for 10 minutes. The cells on the slide are stained with propidium iodide (PI) of 20 μg/ml and subjected to microscopic examination. The relative DNA tail movement in FIG. 22B is represented by the length of DNA tail in FIG. 22A, and the length of DNA tail is estimated by CometScore software. The 1 μM BrdU and the 1 μM BrdU-14 keV NR treatment in FIGS. 22A and 22B are similar to the experiment procedure in FIG. 9A. The BrdU-RS2000 and the RS2000 treatment in FIGS. 22A and 22B are similar to the experiment procedure in FIG. 21A. FIG. 22A shows when FaDu cells is treated with NR and BrdU, FaDu cell has a significant part of blurred region that represents the largest relative DNA tail movements among FaDu cells treated with BrdU-RS2000 or RS2000. FIG. 22B is a quantified result of FIG. 22A. FIGS. 22A and 22B show the combination of NR and BrdU induces DNA damage in FaDu cells, and the combination induces more DNA damage then NR or RS2000, when both of the radiations are used alone.

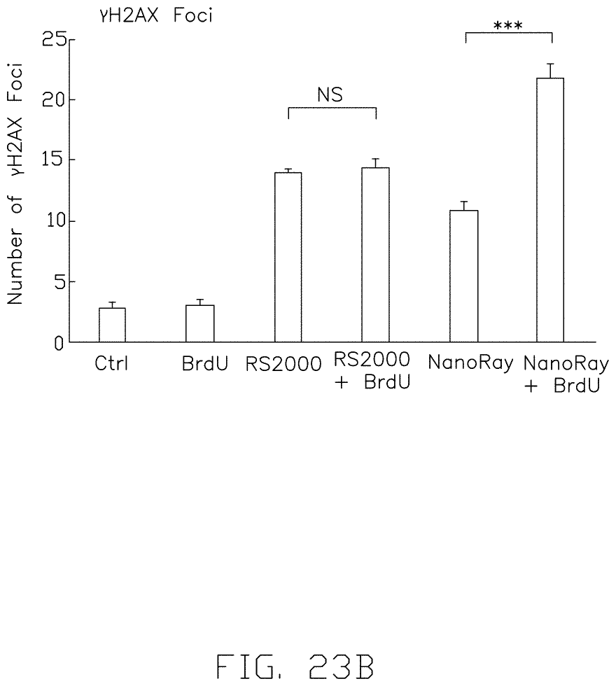

Referring to FIGS. 23A and 23B, p-γH2AX immunofluorescent staining for evaluating DNA damages induced by BrdU, RS2000, BrdU with RS2000, NR, and NR with BrdU, are provided in accordance with exemplary embodiments of the present disclosure. γH2AX is the histone H2AX protein with phosphorylated amino acid on serine 139. γH2AX is a specific marker for DNA DSBs. FaDu cells are seeded on a chamber slides, and are fixed with −20° C., 100% ethanol under room temperature for 15 minutes. The fixed chamber slides are washed with PBS, and permeablized with 0.1% Triton X-100 for 10 minutes. Then, the chamber slides are blocked with 5% milk in PBS for 30 minutes at room temperature, and incubated with primary antibody (anti-γH2AX) overnight at 4° C. FaDu cells in the chamber slides are then washed with PBS, and incubated with secondary antibody which is conjugated by a fluorescent dye for 1 hour at room temperature. The nuclei were countestained with 5 μg/ml of DAPI (4′,6-diamidino-2-phenylindole). The stained FaDu cells are then being mounted and subjected to microscopic examination with a confocal laser scanning microscope (ZEISS LSM780). The number of p-γH2AX are quantified using Metamorph software. FIG. 23A shows a significant amount of γH2AX expression can be observed when FaDu cells is treated with NR and BrdU. FIG. 23B is a quantitative result of FIG. 23A. FIGS. 23A and 23B show the combination of NR and BrdU induces DNA damage in FaDu cells, and the combination induces more DNA damage then NR or RS2000, when both of the radiations are used alone. FIGS. 22A-22B, 23A-23B confirm the enhanced DNA damage in cancer cells is a direct consequence of combining monochromatic X-ray and heavy element containing heavy atom carriers.

The embodiments shown and described above are only examples. Many details are often found in the art such as the other features of the Auger molecular therapy. Therefore, many such details are neither shown nor described. Even though numerous characteristics and advantages of the present disclosure have been set forth in the foregoing description, together with details of the structure and function of the present disclosure, the present disclosure is illustrative only, and changes may be made in the detail. It will therefore be appreciated that the embodiment described above may be modified within the scope of the claims.

What is claimed is:

1. A method for treating cancer in a subject, comprising steps of:
a) administering a pharmaceutical composition comprising a therapeutically effective dosage of iododeoxyuridine (IdU) to the subject; and
b) irradiating a monochromatic X-ray to the subject.

2. The method according to claim 1, wherein the pharmaceutical composition further comprises a pharmaceutical carrier for facilitating absorption and distribution of IdU in the subject.

3. The method according to claim 1, wherein IdU is administered to the subject through a local route or a systemic route.

4. The method according to claim 3, wherein the local route is a topical route, an intratumoral injection, or an intravascular injection.

5. The method according to claim 3, wherein the systemic route is a gastrointestinal route, an intravenous injection, an intravenous drip infusion, an intramuscular injection, or a subcutaneous injection.

6. The method according to claim 1, wherein a radiation dose of the monochromatic X-ray irradiated to the subject is 1 Gy-9 Gy.

7. The method according to claim 6, wherein the radiation dose of the monochromatic X-ray irradiated to the subject is 4.5 Gy or 9 Gy.

8. The method according to claim 1, wherein the therapeutically effective dosage is an injection amount of 3.75 mg-62.5 mg.

9. The method according to claim 1, wherein the therapeutically effective dosage is an injection volume of 7.5 ml-125 ml.

10. A combination of a pharmaceutical composition comprising a therapeutically effective dosage of iododeoxyuridine (IdU) and a radiation dose of a monochromatic X-ray for use in the treatment of cancer.

11. The combination according to claim 10, wherein the pharmaceutical composition further comprises a pharmaceutical carrier for facilitating absorption and distribution of IdU in a subject.

12. The combination according to claim 10, wherein the radiation dose of the monochromatic X-ray is 1 Gy-9 Gy.

13. The combination according to claim 12, wherein the radiation dose of the monochromatic X-ray is 4.5 Gy or 9 Gy.

14. The combination according to claim 10, wherein the therapeutically effective dosage is an injection amount of 3.75 mg-62.5 mg.

15. The combination according to claim 10, wherein the therapeutically effective dosage is an injection volume of 7.5 ml-125 ml.

16. A kit comprising:
a) a pharmaceutical composition comprising a therapeutically effective dosage of iododeoxyuridine (IdU); and
b) a monochromatic X-ray device.

17. The kit according to claim 16, wherein the pharmaceutical composition further comprises a pharmaceutical carrier for facilitating absorption and distribution of IdU in a subject.

18. The kit according to claim 16, wherein the therapeutically effective dosage is an injection amount of 3.75 mg-62.5 mg.

19. The kit according to claim 16, wherein the therapeutically effective dosage is an injection volume of 7.5 ml-125 ml.

20. The kit according to claim 16, wherein the monochromatic X-ray device has an energy of 33 keV.

21. A use of a combination of a pharmaceutical composition comprising a therapeutically effective dosage of iododeoxyuridine (IdU) and a radiation dose of a monochromatic X-ray for the manufacture of a medicament for the treatment of cancer.

22. The use according to claim 21, wherein the pharmaceutical composition further comprises a pharmaceutical carrier for facilitating absorption and distribution of IdU in a subject.

23. The use according to claim 21, wherein the radiation dose of the monochromatic X-ray is 1 Gy to 9 Gy.

24. The use according to claim 23, wherein the radiation dose of the monochromatic X-ray is 4.5 Gy or 9 Gy.

25. The use according to claim 21, wherein the therapeutically effective dosage is an injection amount of 3.75 mg-62.5 mg.

26. The use according to claim 21, wherein the therapeutically effective dosage is an injection volume of 7.5 ml-125 ml.
